(19) United States
(12) Patent Application Publication
(10) Pub. No.: US 2021/0001144 A1
(43) Pub. Date: Jan. 7, 2021

(54) Lumbar Laser Traction and Lumbar Laser Decompression Therapy

(71) Applicants: Scot Leslie Johnson, Tampa, FL (US); Saleem Musallam, Tampa, FL (US)

(72) Inventors: Scot Leslie Johnson, Tampa, FL (US); Saleem Musallam, Tampa, FL (US)

(21) Appl. No.: 16/922,732

(22) Filed: Jul. 7, 2020

Related U.S. Application Data
(60) Provisional application No. 62/871,184, filed on Jul. 7, 2019.

(51) Int. Cl. A61N 5/06; A61H 1/02

(52) U.S. Cl. CPC A61N 5/0613; A61N 2005/067; A61H 1/0229; A61H 1/0222

(57) ABSTRACT

A laser device for use in spinal therapy. The laser device includes a laser manifold having a plurality of recessions therein. Multiple laser modules are disposed within the laser manifold, with each laser module being disposed within one of the plurality of recessions in the laser manifold. The laser device may be incorporated into a spinal therapy bed or other apparatus. In certain embodiments, the laser device may be attached to a motorized actuator.

### BACKGROUND OF THE PRESENT DISCLOSURE

The human spine consists of four distinct regions of bony vertebra which are (generally) connected by intervertebral discs. These consist of the Cervical, Thoracic, Lumbar, and Sacral regions. The Cervical region consists of locations labeled “C0”, “C1”, “C2”, “C3”, “C4”, C5”, “C6”, and “C7”. The Thoracic region consists of locations labeled “T1”, “T2”, “T3”, “T4”, “T5”, “T6”, “T7”, “T8”, “T9”, “T10”, “T11”, and “T12”. The Lumbar region consists of locations labeled “L1”, “L2”, “L3”, “L4”, and “L5”. For the purposes of traction and decompression therapy of the human spine, the only relevant location of the Sacral region is labeled “S1”.

The vertebra are bone structures which vary according to the segment and region of the backbone. Most vertebra are connected by intervertebral discs which allow slight movement of the vertebra and act as “shock absorbers” for the spine. The structure of the intervertebral discs is complex, however their function is dependent primarily on the nucleus pulposus (the central portion of the intervertebral disc) which performs the function of load distribution within the spine. Intervertebral discs are labeled as, for example, the intervertebral disc located between L3 and L4 labeled “L3-4”.

There is a normal curvature of the spine, the so called ‘S-Curve’. There is a normal ‘lordosis’ (forward curve) of the cervical and lumbar spinal regions. There is a normal ‘kyphosis’ (backward curve) of the thoracic and sacral spine.

### BRIEF DESCRIPTION OF THE DRAWINGS

The foregoing disclosure will be best understood and advantages thereof made most clearly apparent when consideration is given to the following detailed description in combination with the drawing figures presented. The detailed description makes reference to the following drawings:

FIG. 1 shows an illustration of the Skull, Cervical, Thoracic, Lumbar and Sacral spine;

FIG. 2 shows an examination of the Lumbar spine, with adjoining intervertebral lumbar discs;

FIG. 3 shows a human patient placed supine on a treatment bed;

FIG. 4 shows a human patient undergoing manual manipulation of the lumbar spine;

FIG. 5 shows a human patient undergoing manual manipulation of the lumbar spine;

FIG. 6 shows a patient on a tilting bed;

FIG. 7 shows a patient on a configurable bed;

FIG. 8 shows a pelvic harness;

FIG. 9 shows the pelvic harness of FIG. 8 in place on a patient;

FIG. 10 shows a patient wearing the pelvic harness of FIGS. 8 and 9 lying on a treatment bed;

FIG. 11 shows a knee support;

FIG. 12 depicts the patient with pelvic harness applied and a knee support positioned underneath the patient's knees;

FIG. 13 shows the patient of FIG. 12 with additional elements;

FIG. 14 depicts the system of FIG. 13, with the addition of a depiction of the intervertebral discs;

FIG. 15 is a close-up view of the vertebra and intervertebral discs of the system depicted in FIG. 14;

FIG. 16 depicts the patient of FIGS. 13 through 15, with the rotational motor raised to a height sufficient to align the vertebra and intervertebral discs connecting L1 through L4;

FIG. 17 shows a close-up of the lumbar spine of FIG. 16;

FIG. 18 shows a modification of the system of FIG. 16, with the rotational motor applying decompressive force to the lumbar spine;

FIG. 19 is a close-up of the lumbar spine under decompression;

FIG. 20 is a side-by-side comparison of the system of FIG. 14 with that of FIG. 18;

FIG. 21 is a side-by-side comparison of the close-up view of FIG. 15 with that of FIG. 19;

FIG. 22 demonstrates the current state of technology for solid state laser modules;

FIG. 23 illustrates both the focused single point laser emission as well as the left and right sides of the cone of laser light produced by slightly defocusing the laser emission;

FIG. 24 shows a laser manifold;

FIG. 25 illustrates an array of eight laser modules positioned below the laser manifold;

FIG. 26 demonstrates an assembly which is one embodiment of the present invention;

FIG. 27 illustrates an electronic means for controlling the laser modules in one embodiment of the present invention;

FIG. 28 describes the assembly of the system of FIG. 26, the laser control PCB, and an optional battery pack;

FIG. 29 describes the system of FIG. 28, with eight focused laser beams emitting from the eight laser modules installed;

FIG. 30 describes the system of FIG. 28, with eight defocused laser beams emitting from the eight laser modules installed;

FIG. 31 describes components which can be utilized to form a portable housing for the laser system of FIG. 28;

FIG. 32 illustrates exploded views of the laser system of FIG. 28 being secured into the shell formed of the manifold cover and base platform;

FIG. 33 shows the system of FIG. 32 fully assembled;

FIG. 34 shows the portable laser device of FIG. 33, with a modification of a port or connection by which a signal may be delivered from outside of the portable laser device;

FIG. 35 demonstrates an example of the profile of the forces applied during both traction and decompression therapy;

FIG. 36 represents the system of FIG. 4, with the addition of the laser module of FIG. 33;

FIG. 37 represents the system of FIG. 6, with the addition of the laser module of FIG. 33;

FIG. 38 represents the system of FIG. 7, with the addition of the laser module of FIG. 33;

FIG. 39 represents a portion of the pelvic harness of FIG. 8, with the addition of a ‘laser slot’ in the rear of this portion;

FIG. 40 represents the system of FIG. 18, with the addition of the laser system of FIG. 33;

FIG. 41 depicts a treatment bed, with upper and lower mattresses;

FIG. 42 represents the system of FIG. 41, with the lower mattress secured to the lower mattress bed support;

FIG. 43 is a close-up of FIG. 42, centered on the laser slot and laser system of FIG. 28;

FIG. 44 represents a patient in traction or decompression as was described in FIG. 18;

FIG. 45 represents a modified version of the laser system module of FIG. 28;

FIG. 46 represents one embodiment of the present invention which may be utilized to indicate the position of the laser system of FIG. 45;

FIG. 47 represents a modified treatment bed in one embodiment of the present invention;

FIG. 48 is the system of FIG. 47, where the laser system of FIG. 45 has been moved backwards fully towards the L5 region;

FIG. 49 is a full view of the system of FIGS. 47 and 48, with the addition of a modified lower mattress mounted to the lower mattress bed support;

FIG. 50 provides a close-up view of the system of FIG. 49;

FIG. 51 represents one preferred embodiment of the present invention; and

FIG. 52 shows a modified patient gown.

### DETAILED DESCRIPTION OF THE DISCLOSURE

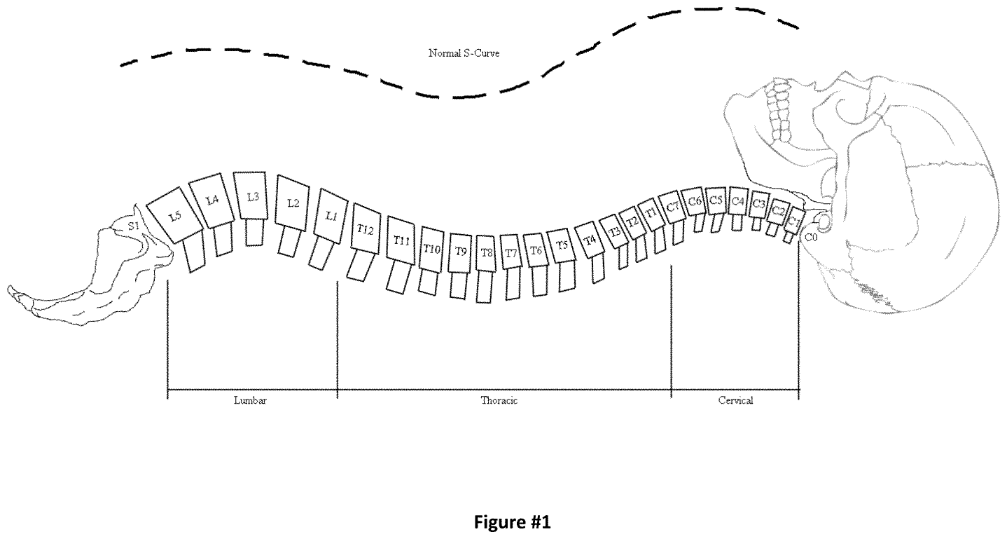

Refer to FIG. 1 for an illustration of the Skull, Cervical, Thoracic, Lumbar and Sacral spine. The normal S-curve is indicated, as are the regions of the spine. Each vertebral bone relevant to traction and decompression therapy is labeled.

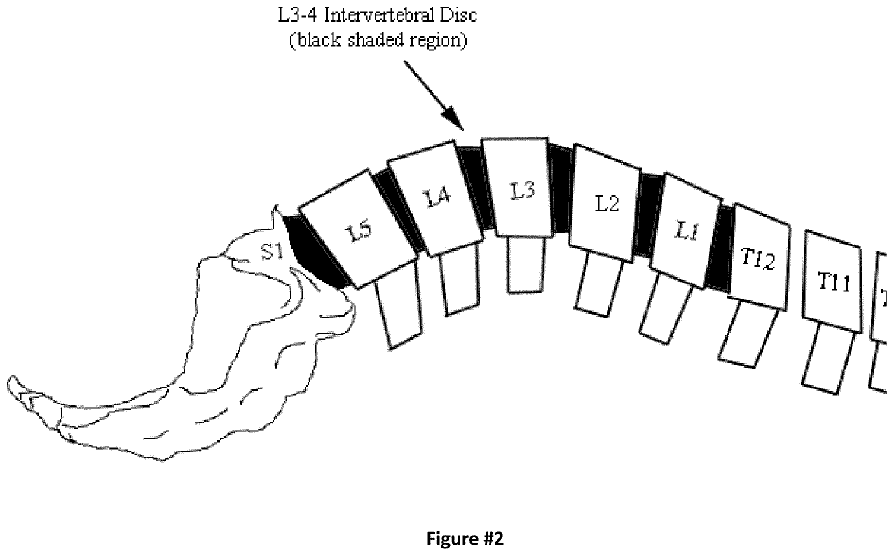

Refer to FIG. 2 for an examination of the Lumbar spine, with adjoining intervertebral lumbar discs. The intervertebral discs are indicated by shaded-BLACK structures (an example of which is pointed out, the intervertebral disc connecting L3 and L4).

Various non-optimal states of the spine may be treated with either traction or decompression. Regarding compressive forces normally experienced by the spine, the intervertebral discs rely on the annulus fibrosus (an outer ring of the intervertebral disc) and the nucleus pulposus which distributes compressive forces in all directions to minimize the compression. The annulus fibrosus consists of fibrocartilage surrounding the nucleus pulposus which distributes compressive forces in normal day-to-day use. The annulus fibrosus in and of itself functions to withstand some portion of compressive forces in normal day-to-day use. There are several conditions which can reduce the (anti-compressive) function of the annulus fibrosus and nucleus pulposus.

‘Herniation’ is the term which describes deformation of the annulus fibrosus which allows the gel-like nucleus pulposus to protrude, distorting muscular function and/or putting pressure on nearby nerves. Commonly referred to as ‘slipped disc’, the disc is not physically slipped. It may bulge, usually in one direction. By unloading the intervertebral disc, the bulge may be pulled back into position and the intervertebral disc can heal and function normally.

‘Degeneration’ is a condition in which the intervertebral discs, either through repeated stress injuries, aging, or genetic predisposition, reduce function of the intervertebral disc and can lead to back pain.

While there are a variety of conditions which lead to back pain, including herniation and degeneration, restoration of the function of the intervertebral disc(s) through physical therapy, traction and decompression can and often does lead to healing of the spine and normal spinal function with regards to day-to-day function and quality of life.

In the case of traction, a continuous distractive force is applied to the spine, and in the case of the present invention, the lumbar spine, to reduce intervertebral disc compressive force(s) and allow for a period of unloading for the intervertebral discs to heal. This technique has certain drawbacks. The paraspinal muscles, those muscles that connect the spinal vertebra, tend to contract over time during traction therapy which lessens and may even exacerbate the condition of the spine. Great care must be taken by either or both the patient (as this form of therapy is readily available to patients for self-treatment) and the healthcare provider for patients seeking treatment.

In the case of spinal decompression, there are two philosophies that exist. The first philosophy, which for the purpose of this provisional patent application will be referred to as ‘basic decompression’, relies on the cyclical application of decompressive forces applied to the spine. In this respect, the decompressive forces applied to the lumbar spine are cycled between higher levels of decompressive forces and lesser applied force (or alternatively periods of zero force). Refer to FIG. 35, described later in this provisional patent application. Rather than applying continuous decompressive force, the decompressive force is applied for a period of time, then relaxed, then reapplied, the cycle being repeated during the treatment. The advantage of basic decompression is the decompressive force applied is temporary, then relaxed, thereby reducing the likelihood that the paraspinal muscles will contract and limit or prevent further unloading of the intervertebral disc.

The second philosophy that is applied to decompression therapy involves both cyclical decompressive force and specific alignment of the lumbar vertebra/intervertebral discs such that decompressive forces applied pull ‘through’ and to the specific disc or discs that are to be treated. For the purpose of this provisional patent application, this form of decompression will be referred to as ‘true decompression’. True decompression therapy represents the most advanced non-surgical form of unloading of compressive forces and healing of the intervertebral disc(s).

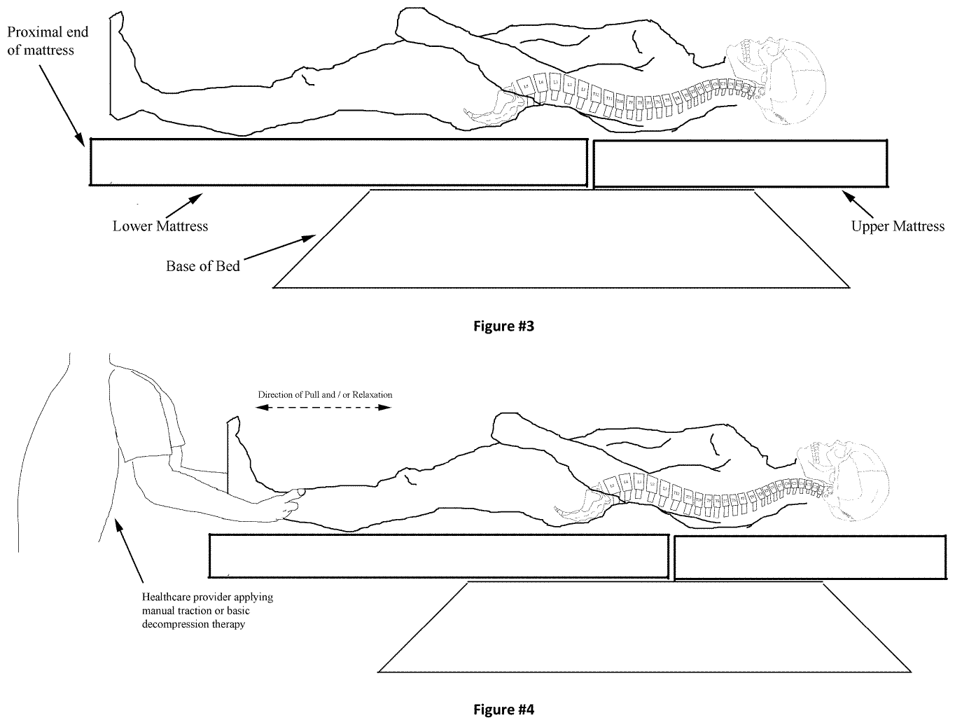

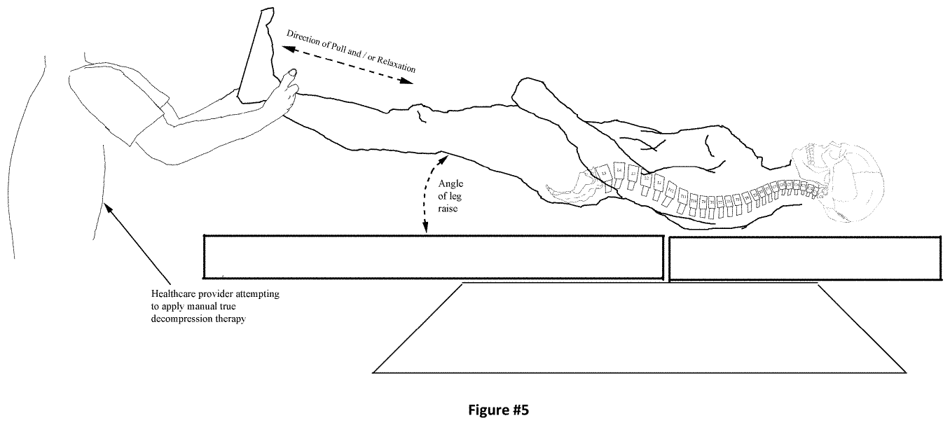

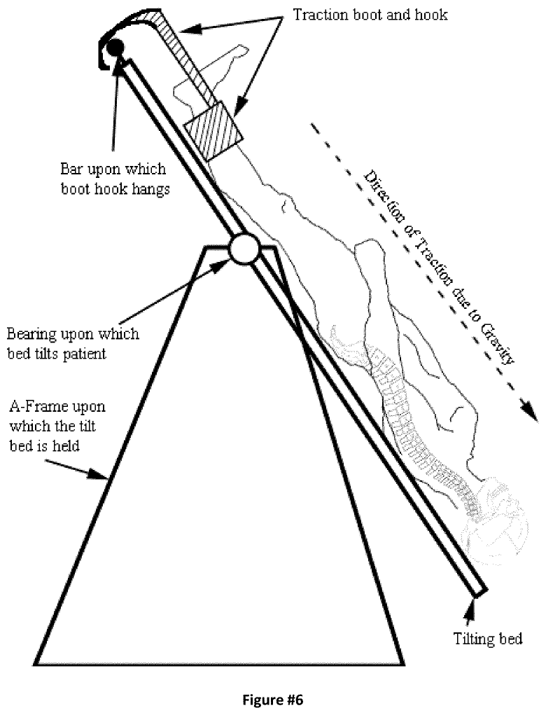

Typically, the human patient is placed supine on a treatment bed (refer to FIG. 3). The most basic form of lumbar traction or decompression therapy relies on “manual” manipulation of the lumbar spine (refer to FIGS. 4 and 5). The healthcare provider typically grabs the patient by the ankles or lower legs. The healthcare provider then pulls the ankles or lower legs in an attempt to provide traction, or cycles the pulling force in an attempt to provide basic decompression therapy. Indeed, manual manipulation can be applied in a way to provide ‘true decompression’, though given the ability of the healthcare provider to maintain elevation of the legs and pelvis and to reliably cycle tension, manual manipulation for true decompression is not generally realistic. As will be described further in this application, true lumbar decompression therapy almost always involves a medical apparatus to maintain a certain posture of the lumbar spine, and to apply consistent tension cycles.

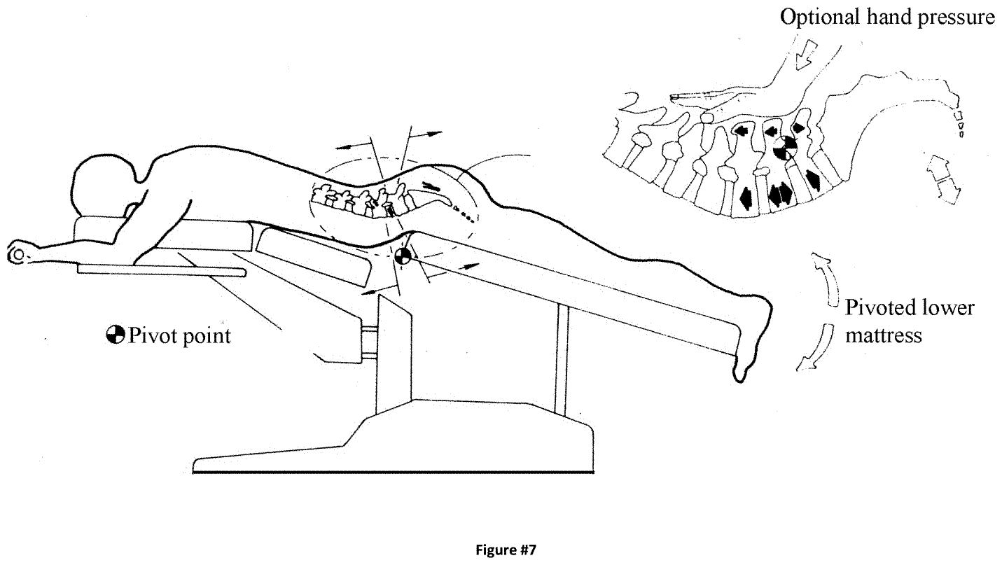

Various mechanical apparatus have been developed to aid in either traction or decompression therapy. A simple, basic method of lumbar (and perhaps fully spinal) traction is the use of ‘traction boots’. In one application, a bar is secured between two stable posts, perhaps a door frame. The patient applies the traction boots, a wrap or boot that is applied firmly about the ankles and/or feet, the boots having some form of hook. The patient then contorts themselves such that the hooks of the boot are hanging on the bar, the patient then suspended by the boots on the bar and gravity provides the traction force on the spine. In another embodiment, an A-frame structure containing a hinge or rod-on-bearings at its apex upon which a bed or platform is attached forms the apparatus. The patient attaches the aforementioned ‘traction boots’ to themselves. The patient then rotates the platform to the standing position, stands upon the platform and aligns the hooks of the traction boots to a bar located at the base of the platform such that the hooks of the traction boots are upon the platform's bar. Then, the patient physically rotates the platform upon which they lay backwards, such that their head is at least partially below their body, gravity providing the force which unloads their spinal segments (refer to FIG. 6).

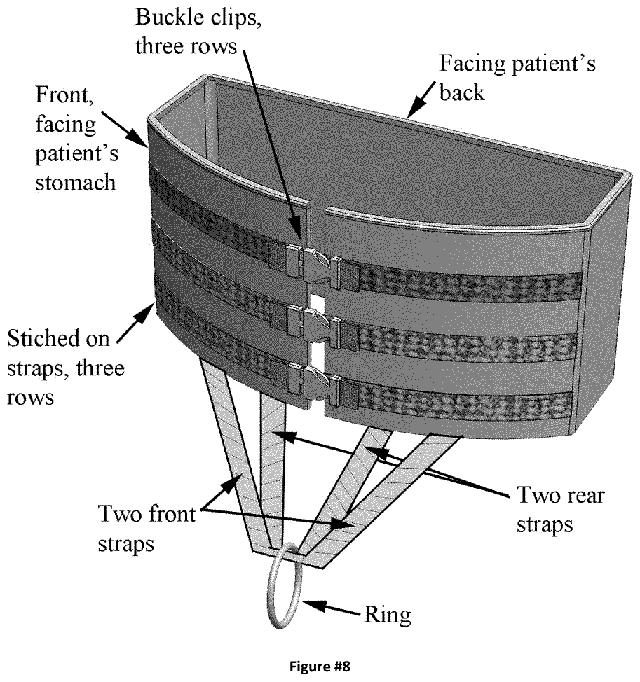

An additional means by which traction, basic decompression, and limited true decompression of the lumbar spine may be accomplished is through the use of a manually configurable or automated bed upon which a patient lay prone (on their stomach, face down). Refer to FIG. 7. The patient lay positioned such that the lowest mattress can pivot up or down (the upward limit being parallel to the floor upon which the overall device is mounted). The healthcare provider must lay the patient on the apparatus such that the intervertebral disc location to be treated is located relative to the pivot point. In one method, the healthcare provider can apply manual pressure upon the lumbar spine to decompress the intervertebral disc to be treated. In another aspect, the pivot point of the lowest mattress may be rotated downwards and upwards, in a way to apply a limited decompression of the lumbar spine (this can be accomplished by an appropriate means, such as a rotational motor at the pivot point, a linear motor attached to both the lowest mattress and a stationary point within the stationary base of the apparatus, or other means).

More advanced apparatus have evolved to provide true lumbar decompression in a controlled and sustainable manner.

In one embodiment, a pelvic harness is applied to the patient, the patient then lay supine on a treatment bed, and a motor is connected to an attachment means on the pelvic harness. The motor is then raised or lowered such that the pelvic harness may be raised, tilting the pelvic harness about the lumbar spine and aligning certain of the lumbar vertebra and intervertebral discs. The motor then applies cyclical decompressive forces to the aligned spine, providing true lumbar decompression.

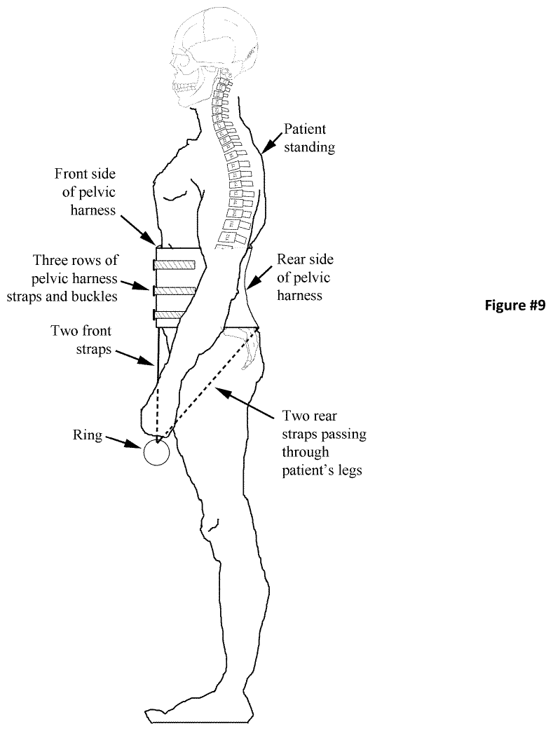

Refer to FIG. 8. In one embodiment, a pelvic harness is described, wherein a wide textile ‘wrap’ is meant to encompass the circumference of the patients lower back. Three straps secured to the textile wrap tighten and secure the wrap about the patient, secured with buckle clips (in this case ‘Fastex’ style clips) at the front of the patient's body. Two straps extend from either side of center at the bottom of the rear portion of the wrap (that portion facing the patient's back), and two straps extend from either side of center at the bottom of the front facing portion of the wrap (that portion facing the patient's stomach). The straps extend to an attachment point at a ring (in this case a stainless steel ring).

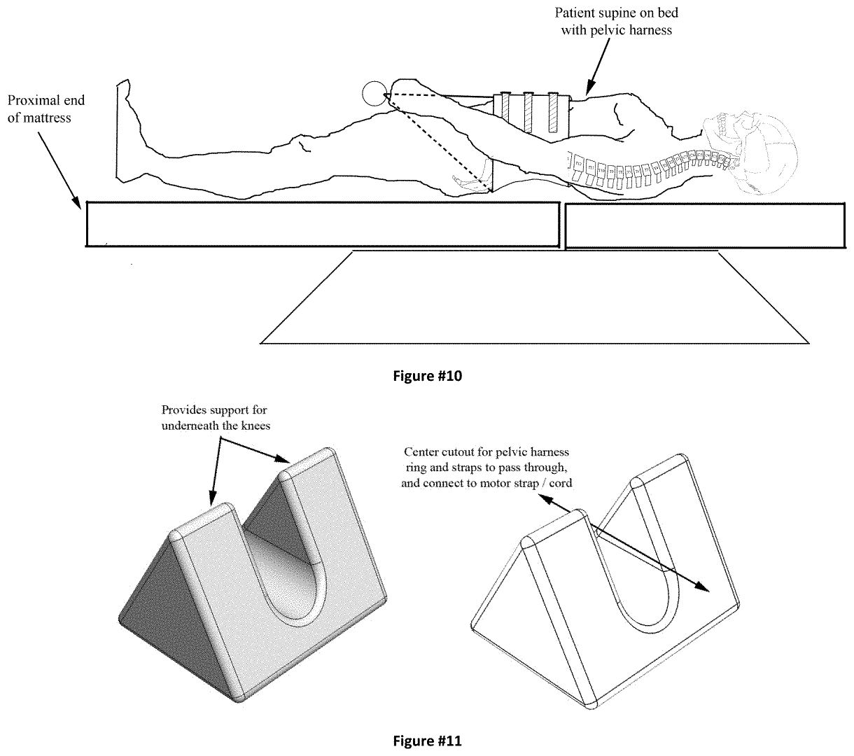

In one method of applying the pelvic harness, the patient is standing and the pelvic harness (with the three buckles disengaged) is applied to the patient. First, the patient steps through either the left or right opening provided by the front and rear straps on that side. For example the patient may extend their right foot/leg through the right side front and rear straps, and then likewise their left foot/and leg through the left side front and rear straps, such that the ring hangs between their legs. The textile wrap is then positioned appropriately about the patient's lower back, and is secured there by tightening and buckling the three securing straps. Refer to FIG. 9.

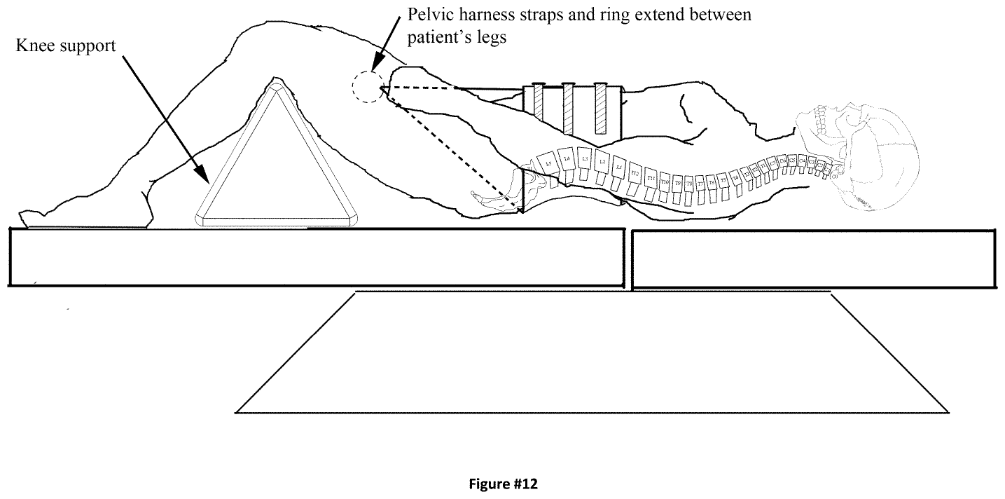

Refer to FIG. 10. With the pelvic harness secured to the patient, the patient is then placed supine on a treatment bed, their feet facing the proximal end of the bed as indicated in the Figure. As briefly described previously, cyclical decompressive forces will ultimately be provided during treatment. The weight of the patient and the friction of the mid and upper body against the treatment bed may be sufficient to provide opposition to the decompressive forces such that the patient's body would not ‘slide’ towards the decompressive force's origin. In some embodiments, arm posts (positioned such that the patient's underarms and the arm posts are coincident) located in the upper mattress (not shown) would provide additional opposition to the decompressive forces applied sufficient to prevent the patient body from sliding towards the decompressive force origin. In some embodiments, an upper body harness secured to the patient's upper body and with a connection to the distal end of the bed (near the patient's head) would provide additional opposition to the decompressive forces applied sufficient to prevent the patient body from sliding towards the decompressive force origin. However the patient's body is secured such that the patient body does not slide towards the decompressive force origin, the decompressive forces applied to the patient will have limited or no ability to decompress the lumbar spine if the patient's body slides towards the decompressive force origin.

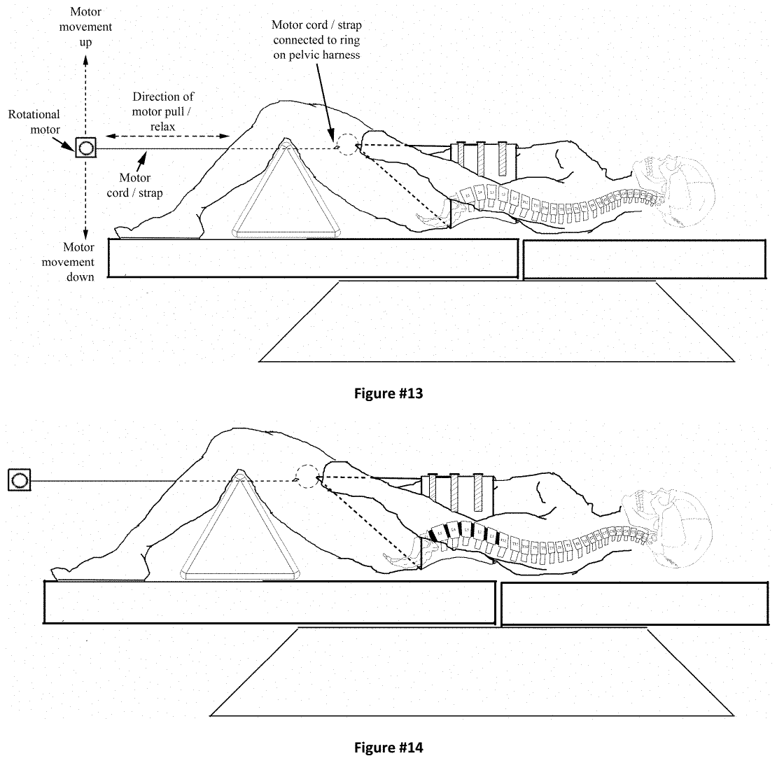

FIG. 11 depicts one example of a knee support. The knee support in one embodiment my provide a means to reduce the lordosis of the lumbar spine and thus reducing the amount of height the decompressive force means needs to be raised to align the lumbar vertebra/intervertebral discs. The knee support provides additional comfort to the supine patient. As shown in the Figure, a center channel is provided such that the ring and straps between the patient's legs may be accessed by the healthcare provider for connection to a decompressive force means.

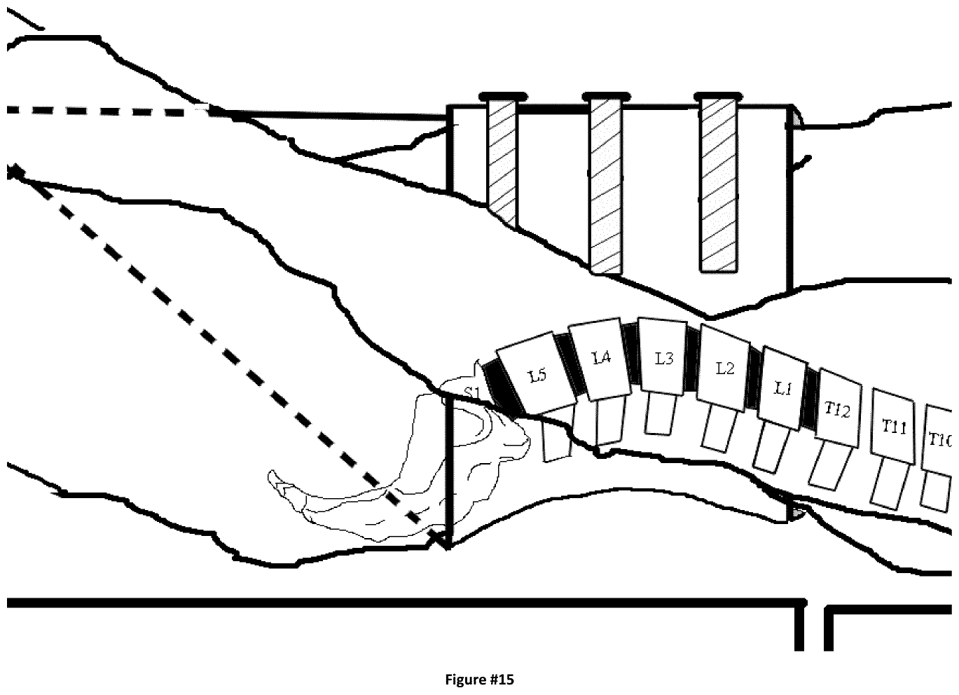

FIG. 12 depicts the patient with pelvic harness applied, the knee support positioned underneath the patient's knees. The pelvic harness straps and ring are shown (dashed lines) residing between the patient's legs. In this Figure, the full patient spine is shown.

As previously described, ‘true decompression’ therapy involves both cyclical decompressive force and specific alignment of the lumbar intervertebral discs/vertebra such that decompressive forces applied pull ‘through’ and to the specific disc or discs that are to be treated.

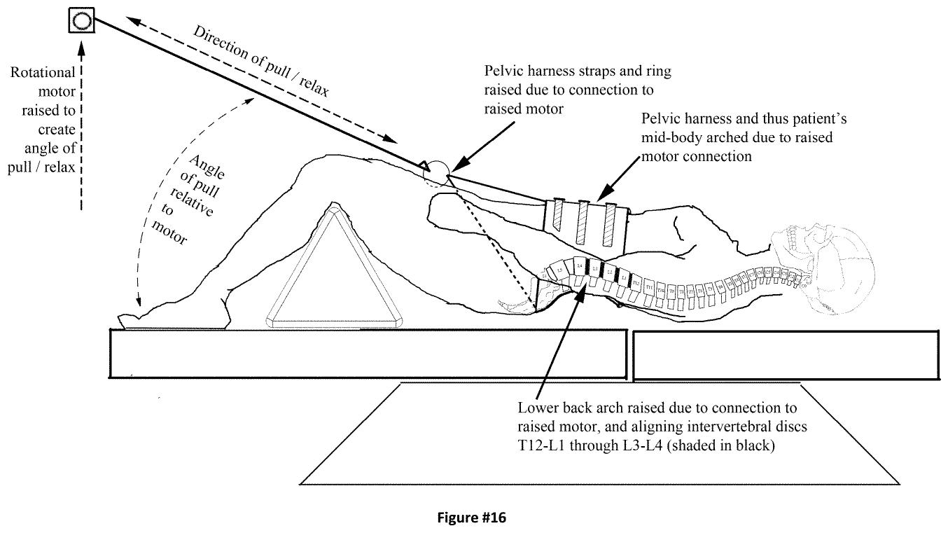

Refer to FIG. 13. The patient/system of FIG. 12 is shown, with additional elements. One decompressive force means, a ‘rotational’ motor, is shown near the proximal end of the treatment bed. The rotational motor contains a motor strap or cord which is extended to a connection to the ring of the pelvic harness. This cord may pass through the center portion of the knee support. The rotational motor may be moved upwards or downwards to change the alignment of the pelvic harness and thus the lumbar vertebra and intervertebral discs. Via the rotational motor, the motor strap or cord can provide a decompressive ‘pull’ or corresponding relaxation of the decompressive forces.

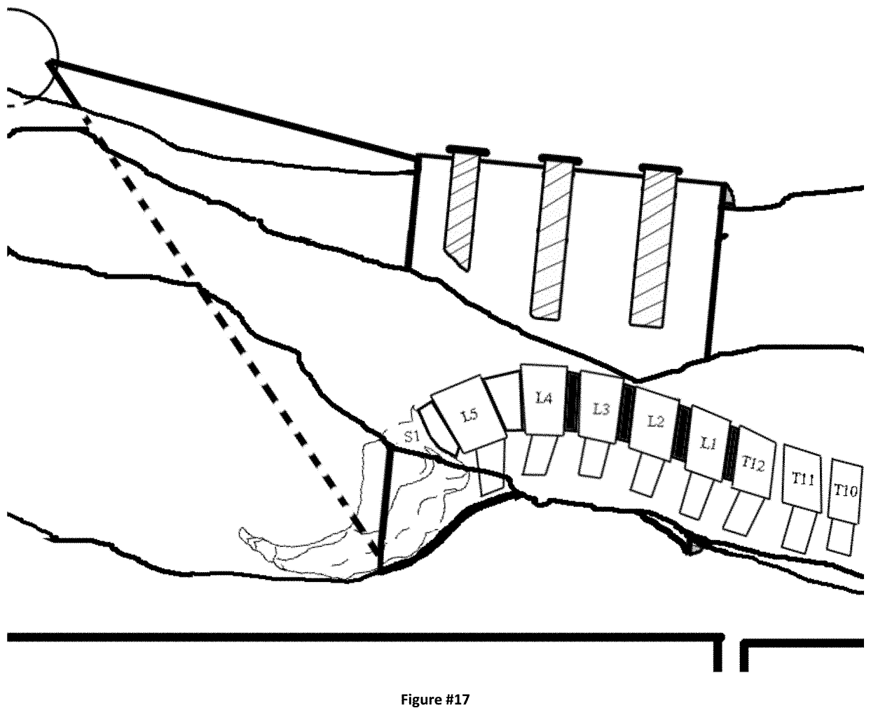

FIG. 14 depicts the system of FIG. 13, with the addition of a depiction of the intervertebral discs (shaded in BLACK) relative to lumbar decompression therapy. The lumbar intervertebral discs are shown within the normal lordosis of the lumbar spine, which may be reduced due to the application of the knee support.

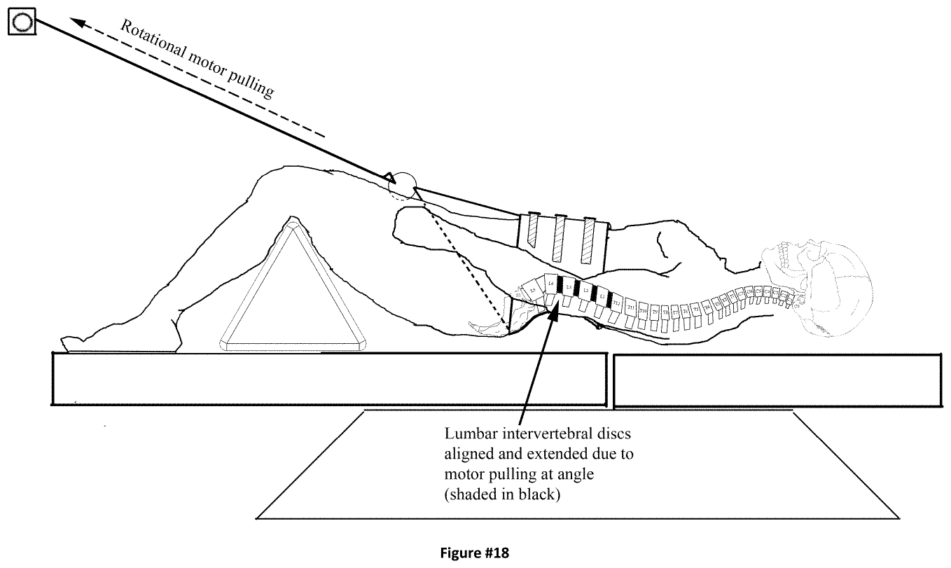

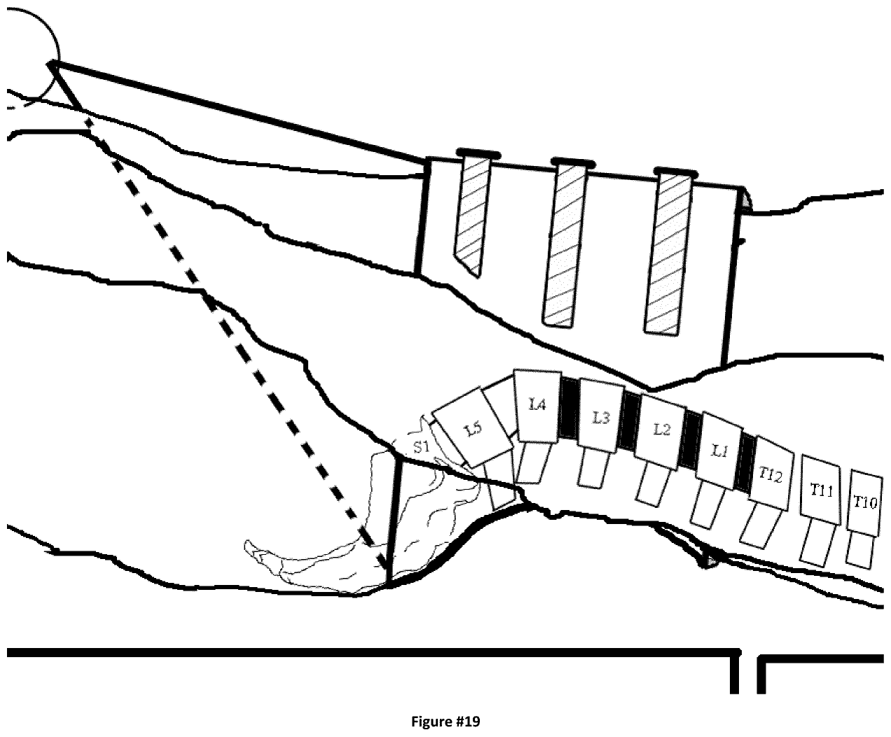

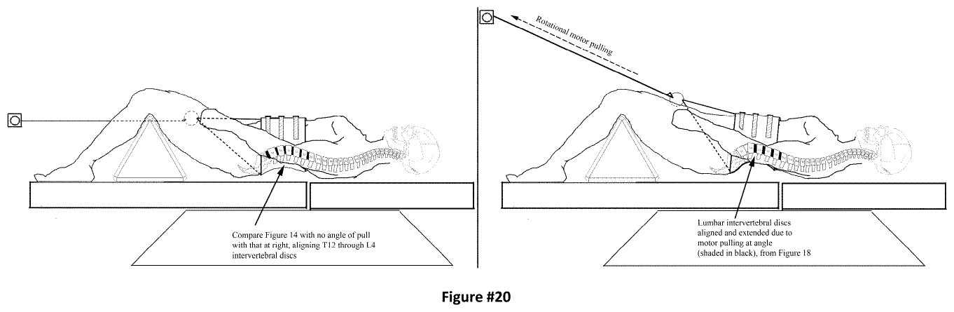

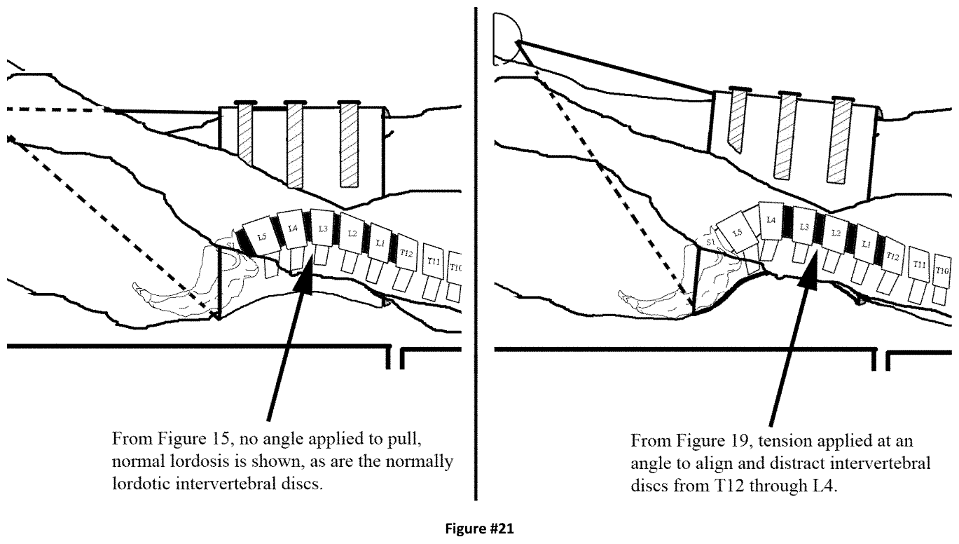

FIG. 15 is a close-up view of the vertebra and intervertebral discs of the system depicted in FIG. 14.

‘True decompression’, as stated previously, requires both a cycling of decompressive forces and alignment of the vertebra/intervertebral discs such that decompressive forces pull those aligned discs. Of further note, cycling of decompressive forces allows for a partial regression towards lordosis during the low tension or no tension phase of the cycle. It is known that the intervertebral discs draw in fluids through flexion (a result of differences in interdiscal pressure). Thus, as the vertebra and intervertebral discs are allowed to slightly flex between alignment and at least partial return towards lordosis, the intervertebral discs are allowed to further rehydrate, restoring intervertebral disc function.

FIG. 16 depicts the patient of FIGS. 13 through 15, with the rotational motor raised to a height sufficient to align the vertebra and intervertebral discs connecting L1 through L4 (aligned intervertebral discs shaded in BLACK). The rotational motor is shown raised from its previous position (of FIGS. 13 through 15), creating an angle of pull (described in the Figure). The motor cord or strap and the direction of pull or relaxation is shown, the motor cord or strap connected to the ring of the pelvic harness. In this embodiment, the pelvic harness straps connected to the ring are elevated in line with the motor cord or strap, which rotates the pelvic harness in a likewise manner. The rotated pelvic harness reduces the lordosis of the lumbar spine, the angle illustrated sufficient to align the vertebra (and thus the intervertebral discs) L1 through L4. In this embodiment, the affected intervertebral disc to be treated is L3-4.

FIG. 17 shows a close-up of the lumbar spine of FIG. 16, to illustrate the aligned intervertebral discs shaded in BLACK.

FIG. 18 is a modification of the system of FIG. 16, the rotational motor applying decompressive force to the lumbar spine. In the Figure, intervertebral discs connecting L1 through L4 (aligned intervertebral discs shaded in BLACK) are unloaded and elongated.

FIG. 19 is a close-up of the lumbar spine under decompression, to illustrate the aligned and elongated intervertebral discs shaded in BLACK.

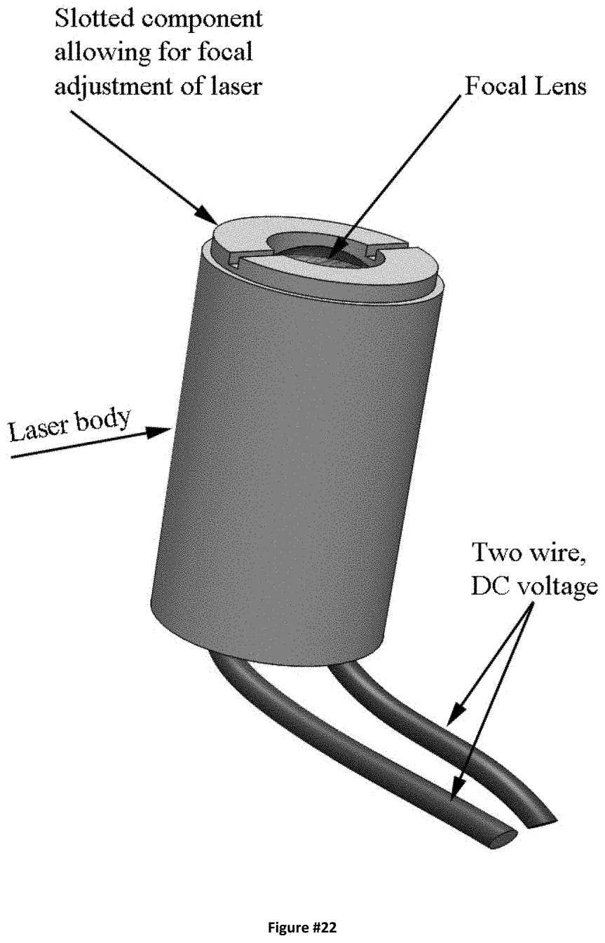

FIG. 20 is a side-by-side comparison of the system of FIG. 14 with that of FIG. 18.

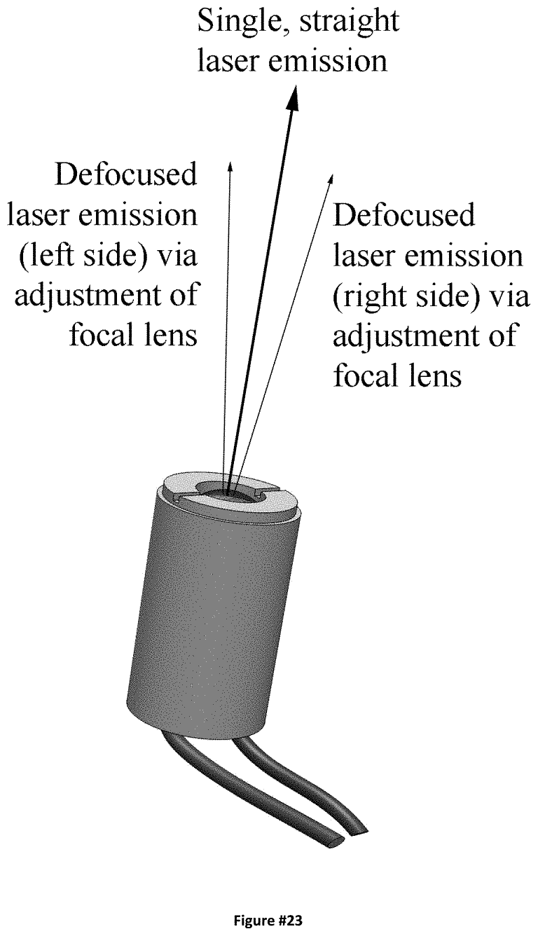

FIG. 21 is a side-by side comparison of the close-up view of FIG. 15 with that of FIG. 19.

Lumbar Laser Traction and Lumbar Laser Decompression Therapy

It is well demonstrated in the peer-reviewed, published clinical literature that wavelengths in the so-called ‘optical window’, ranging from roughly 600 nm to approximately 940 nm penetrate the skin (when applied externally to the body) and affect patient tissue and blood. The effects range from upregulating adenosine triphosphate (ATP) production, ATP being the energy currency of the body. In states of dysregulation, wound repair, fatigue and other diseases, a substantial amount of the body's normal ATP production is devoted to healing. Light therapy within these wavelength windows has been shown to be absorbed (the photonic energy) by certain chromophores, fluorophores, flavins and other structures which translates to increases in the respiratory chain which produces ATP, and thus upregulates this process and thus aids both in the healing process and the patient's perceived quality of life. Further well-studied effects of wavelengths in this region include shortened rates of known healing times, improved quality of wound healing, improved rheological properties of the blood, improved oxygen transport, upregulated immune system function, and most importantly reduction of inflammation (for example reduction of pro-inflammatory cytokines, upregulation of anti-inflammatory cytokines, and reduction of pro-inflammatory pre-cursors). Ultimately, the aforementioned effects of light therapy (the light therapy typically generated by either lasers or light emitting diodes [′LED′]) ultimately lead directly and indirectly to reduction in pain.

The depth of penetration of laser wavelengths tends to be shallower towards the 600 nm range than the near infrared range (900 nm). As the lumbar spine is surrounded by paraspinal and other muscular tissue, connective tissue, and other tissue structures, an infrared wavelength may be most desirable for its depth of penetration.

The application of a laser therapy modality to the treatment of the lumbar spine is a common practice. At the time of this writing of this provisional patent application, the application of lumbar spinal traction or lumbar spinal decompression with the simultaneous application of a laser therapy (or generally light therapy) modality is uncommon and to the best of the inventors' knowledge non-existent in the approved-for-marketing medical device market/industry. In one aspect, the simultaneous application of laser therapy with either lumbar traction or lumbar decompression is a convenience for the healthcare provider (not having to expend billable time and effort to provide one therapy followed by another) and for the patient (not having to schedule more time to receive both therapies in series, possibly impacting their work schedule or other responsibilities). In another aspect, the simultaneous application of laser therapy and either lumbar traction or lumbar decompression (basic or true) offers an anatomical benefit. This anatomical benefit takes the form of improved penetration of the photonic laser energy into the intervertebral discs, once elongated. Laser penetration through bone is limited, when compared to penetration through skin/muscle/tissue/blood/etc. When the lumbar spine, in its normal lordotic state, is normally loaded/not aligned lasers placed underneath the lumbar spine (assuming the patient is lying supine on a treatment bed) will affect the properties discussed previously beneficial to the patient, but in a potentially limited way due to the bony vertebra shielding in part the intervertebral discs which are to be treated by either traction, basic decompression, or true decompression. However, as with true decompression (refer to FIG. 21) the proper alignment of the lumbar vertebra and discs L1 through L4 and by application of true decompression (cycling of the unloading force), the elongation of the vertebra and thus the intervertebral discs involved creates an improved scenario for laser penetration into the intervertebral discs, especially when laser wavelengths utilized provide deeper penetration into the affected regions. The unloaded and extended intervertebral discs also unload the nerves/nerve roots, which allows for the reduction of nociceptive signaling (the pain signal) from the site of pain which travels into the spinal cord, through to the amygdala (where pain is interpreted by the brain). The amygdala, receiving a reduced pain signal, will transmit a descending signal to the site of the “injury” (often an impinged nerve/nerve root caused by a bulging disc for example), resulting in less inflammatory pain response.

The nerves/nerve roots exit the spine on either side of center of the spine at each of the vertebra/intervertebral discs. While it may seem advantageous to place a single laser at the center of each location of treatment (within a system), it may be and is the subject of one embodiment of the present invention, to place two lasers, one on either side of the center of the spine. One other aspect of the laser therapy modality, applied either internally or externally which has been reported extensively in the peer-reviewed published literature is the concept of ‘translation’. Translation refers to the concept that the fluids surrounding the tissues/wounds etc. to be treated (for example blood, spinal fluid, etc.) that are exposed to an appropriate wavelength of light and an appropriate intensity of light will carry these effects to both nearby structures and further the entire system of the patient body. Indeed, whole therapeutic modalities exist which are based on the process of translation, for example ultraviolet blood irradiation (the withdrawal of a very small amount of blood, the exposure of that blood to intense ultraviolet light [often either UVC or UVB or both], and the re-introduction of that light into the patient). Ultraviolet blood irradiation (“UBI”) has been established for well over one-hundred years, its original development resulting in a Nobel Prize for the treatment and continuously documented treatment of a variety of diseases, including sepsis and a myriad of other medical disease states.

There is a need for the integration of the light therapy (and in many embodiments presented herein, laser therapy) modality with lumbar traction, basic lumbar decompression, and most importantly and effectively true lumbar decompression. The remainder of this provisional patent application will describe embodiments of these apparatus.

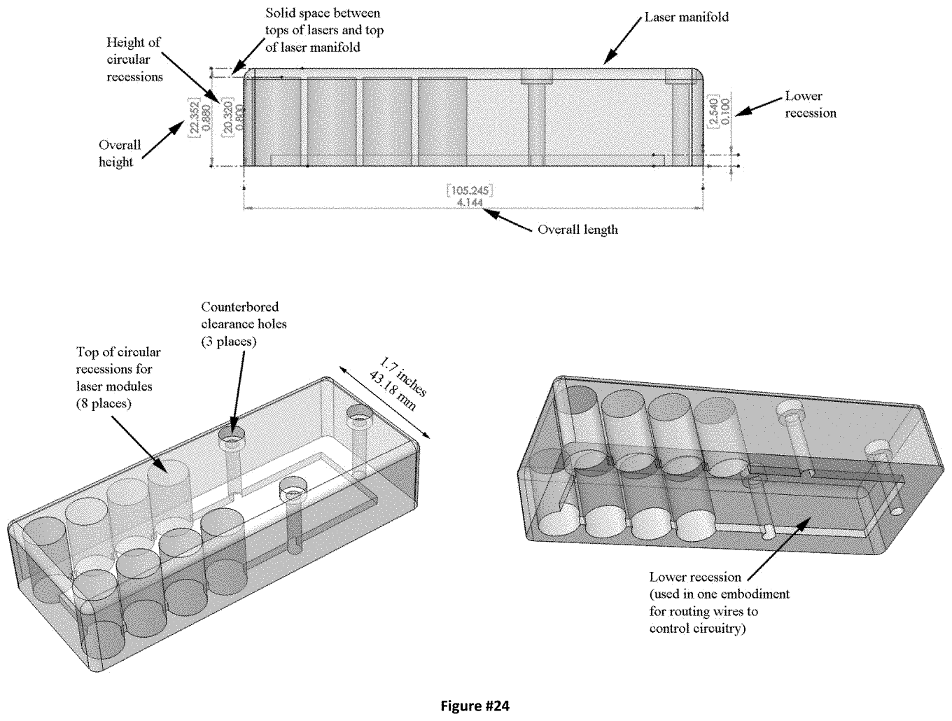

FIG. 22 demonstrates the current state of technology for solid state laser modules. Compact laser diodes of miniature size range in wavelengths from less than 635 nm to greater than 904 nm based on the state-of-the-art laser technology of today. These laser diodes tend to increase in size relative to the optical output intensity (typically given in mW). State-of-the-art laser diode technology is now placed into metallic tubes which tend to contain control circuitry and feedback mechanisms to ensure optical output is maintained. The state-of-the-art control technology allows for the simplest of power connections, namely two wire DC voltage connection, and tends to align with common logic circuit voltage of 3.3 VDC. This system of a contained laser/control circuitry/feedback circuitry in miniaturized form leads directly to one embodiment of the present invention.

In terms of physical presentation, the laser module packages range from less than 6.4 mm in diameter to greater than 10.4 mm. These modules tend to range between 12 mm to greater than 17 mm in length. In terms of optical output, these modules tend to put out less than 1 mW to greater than 200 mW.

There are other variations of the current state-of-the-art laser module packages, such as a small conductive spring placed at the bottom of the laser module, wherein the spring carries the positive voltage signal and the case itself carries the ground signal. However, in the embodiment of the present invention demonstrated in FIG. 22, the two wire system is utilized.

In this embodiment of the present invention, the laser module represented in FIG. 22 is an 808 nm laser wavelength. The optical power output is 50 mW. The optimal operating voltage is 3.3 volts DC (VDC). The physical package is 10.4 mm in diameter and 17 mm in length. A two wire connection is required to turn the laser on. There exists a focal lens aligned over the laser diode, and a slotted component allowing for a focusing mechanism, whereby the laser may be purely a straight beam, or slightly defocused such that instead of a single focal point some distance from the laser, a defocused region of the laser, forming a small ‘circle’ of laser light on the intended target. For example, this type of laser module is produced by US-Lasers, part number M808-50.

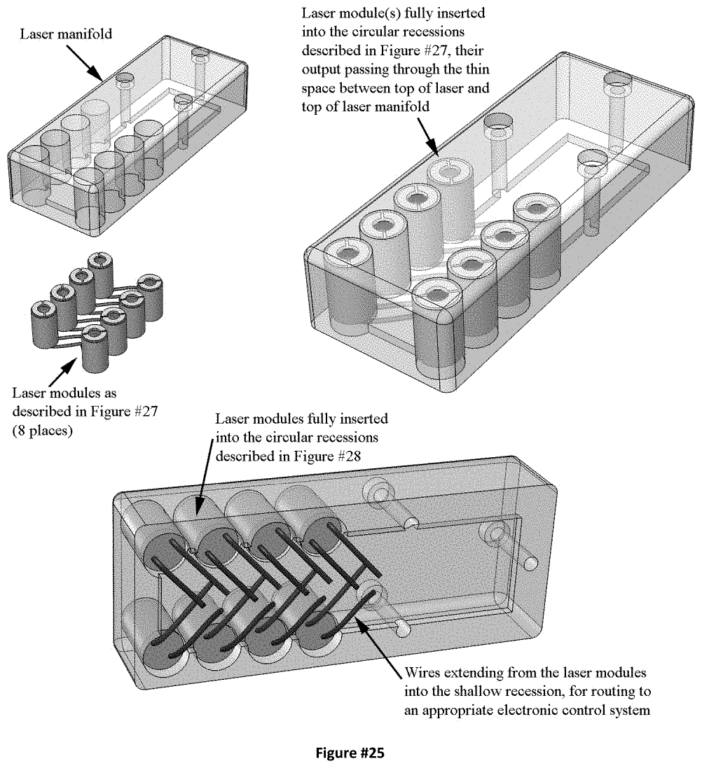

FIG. 23 illustrates both the focused single point laser emission as well as the left and right sides of the cone of laser light produced by slightly defocusing the laser emission.

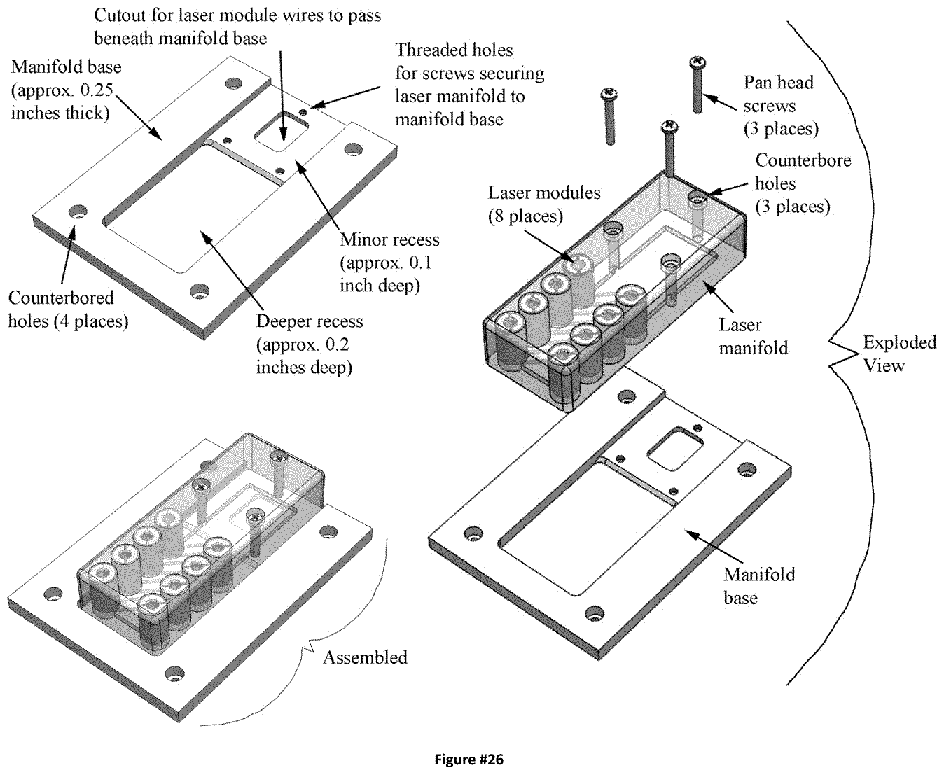

As previously described, one embodiment of the present invention involves placement of two laser modules on either side of a central axis (representing the center of the lumbar spine). FIG. 24 demonstrates a “laser manifold”, a mechanical structure designed to house the laser modules and permit their optical emission to pass through to the lumbar spine. Due to the state-of-the-art laser module mechanical dimensions suggested in one embodiment of the present invention, the mechanical dimensions of the laser manifold are minimal. In this embodiment of the laser manifold, the mechanical dimensions are approximately 4.14 inches in length, 0.88 inches in height, and 1.7 inches in width. Circular recessions exist which extend from the bottom of the manifold up and to 0.08 inches from the top of the laser manifold, and total eight in number. A total of 400 mW of optical energy is provided in this embodiment, derived from the eight 50 mW laser modules.

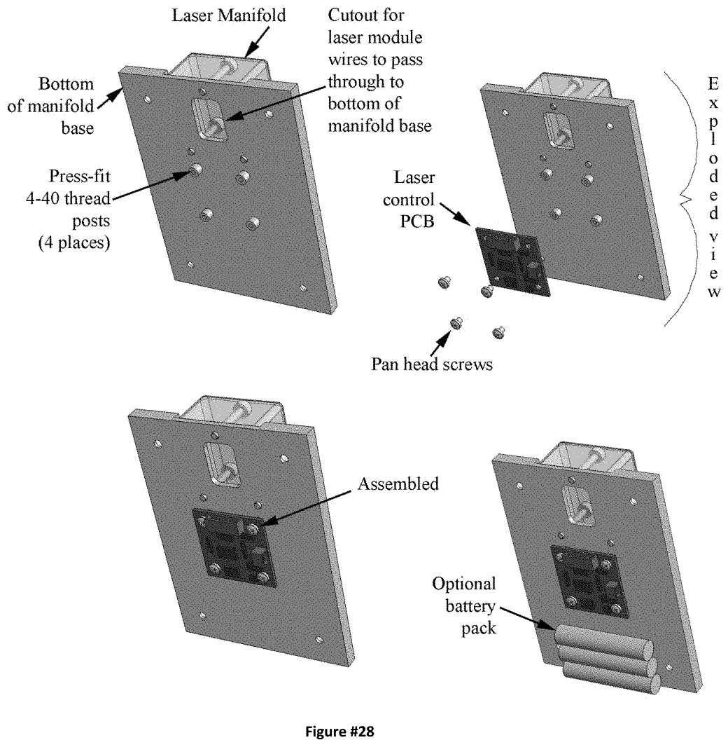

The laser manifold can be injection molded, machined, or by some other means created of a suitable material transparent to the desired wavelength(s) utilized, and in this embodiment this material would be maximally transparent to 808 nm optical energy (ideally between 80% to 100%). It is necessary to polish the space between the top of the circular laser recession and the top of the laser manifold. Three counterbored holes are shown, which can be utilized with appropriate hardware (e.g. screws) to secure the laser manifold to some type of base (shown in FIG. 26). A recession at the bottom of the manifold exists (0.10 inch in depth) which allows for routing the laser module wires to some appropriate electronic control mechanism.

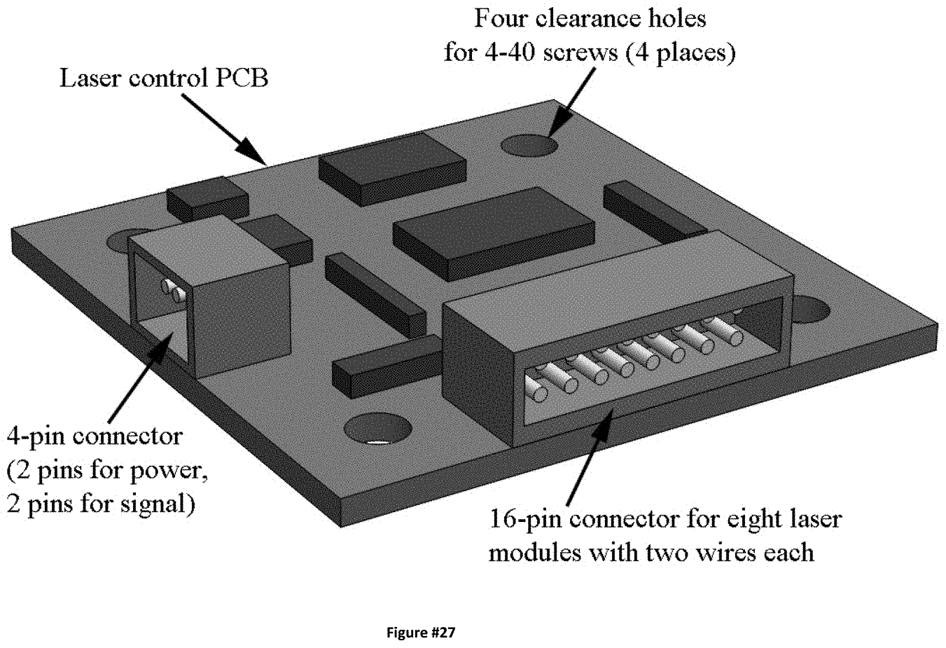

FIG. 25 illustrates an array of eight laser modules positioned below the laser manifold (top left). At top right, the laser modules are shown fully seated within their circular recessions. At the bottom of the Figure, the laser module wires are routed towards the end of the laser manifold which contains the counterbored holes and are contained within the bottom 0.1 inch recession.

FIG. 26 demonstrates an assembly which is one embodiment of the present invention. At top left, a “manifold base”, machined from a suitable, preferably lightweight material such as aluminum or delrin, is shown, which is 0.25 inches thick. A deeper recess is identified, 0.2 inches in depth, which corresponds when mated to the laser manifold the area beneath the laser modules. A minor recess is also identified which corresponds to the portion of the laser manifold which contains the three counterbored holes, and when the laser manifold is mated to the manifold base provides a foundation upon which the laser manifold is secured. Three threaded holes (in this embodiment 4-40 thread) are identified within the minor recessed region. A cutout through the entire manifold base is shown, meant to allow the laser module wires to pass through, for connection to some electronic control means beneath. At right, an exploded view of the assembly of FIG. 26 is shown. Progressing downward from three cross-slotted pan screws, 4-40 thread and 0.875 inches long, the laser manifold is shown (which contains the laser modules) and beneath that, the manifold base. At lower right, the exploded assembly is shown collapsed and completed.

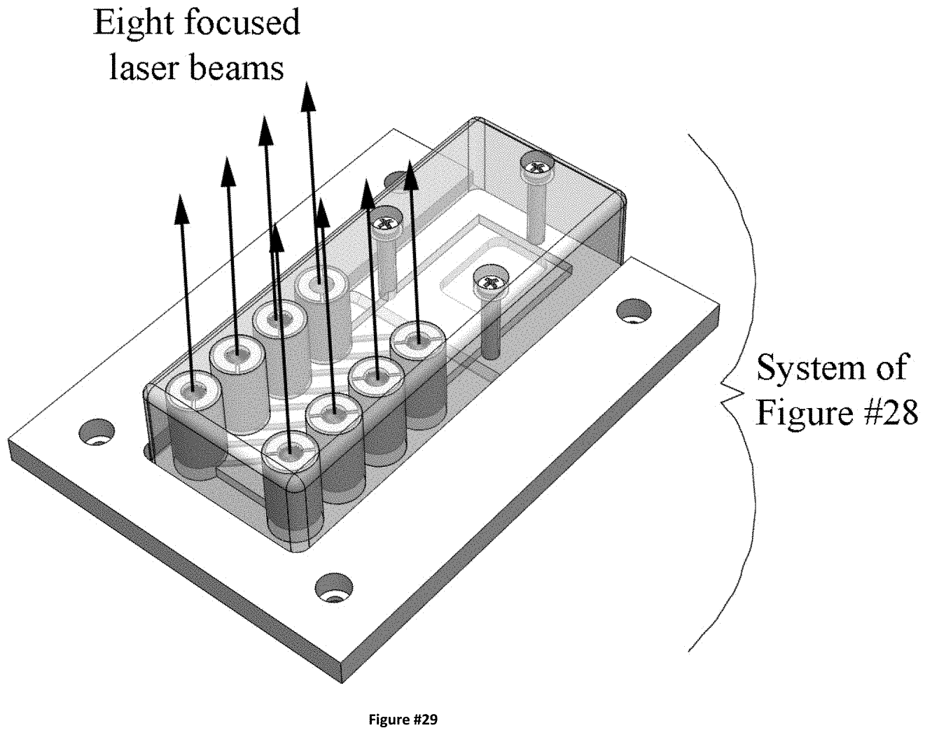

FIG. 27 illustrates an electronic means for controlling the laser modules in one embodiment of the present invention. A printed circuit board (“PCB”) is shown, the ‘laser control PCB’, which is 0.060 inches thick and measures 1.4 inches on either side. Aside from the necessary circuitry required to control the laser modules, the laser control PCB contains in this embodiment a 4-pin connector suitable to receive power (2 pins) and a control signal (2 pins). A control signal may be necessary for external communication for a variety of purposes. In one example, the control signal may be connected to a simple switch, allowing a healthcare provider to turn the laser modules on or off. In another example, the control signal may be connected to a sensor which in some way detects the appropriate conditions to turn the laser modules on or off (for example a pressure sensor). In another embodiment, the laser control PCB may be in communication with another device which, in one of the final embodiments of the present invention, controls not only when the laser modules are on or off but also controls either the traction or decompression therapy described previously in this application, as when lumbar laser traction or lumbar laser decompression are applied simultaneously. In another embodiment, the laser control PCB may be equipped with optical and/or wireless communications to an external control means.

The laser control PCB also contains a 16-pin connector, suitable for connection to the wires extending from the eight laser modules in the system of FIG. 26 (eight laser modules with two wires each). The laser control PCB also contains four holes suitable for clearance of #4 screws.

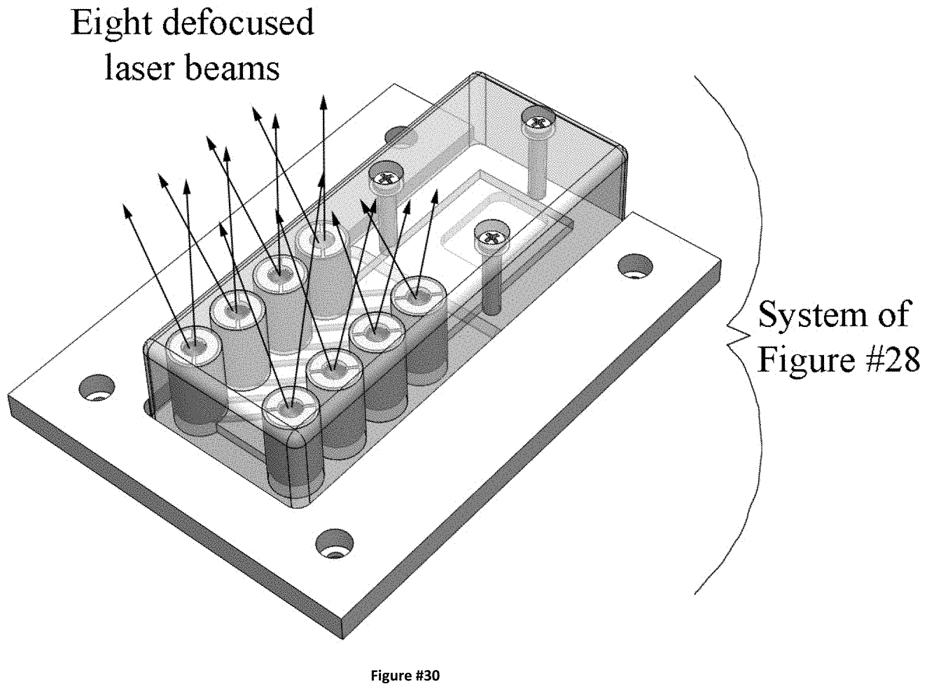

FIG. 28 describes the assembly of the system of FIG. 26, the laser control PCB, and an optional battery pack. At upper left, the bottom of the manifold base is shown, demonstrating the placement of press-fit threaded inserts which in this embodiment are 4-40 threaded and 0.1 inch tall (four places). An exploded view of the assembly is shown at top left, demonstrating four cross-slotted pan head screws (4-40 thread, 0.125 inches long), followed by the appropriate orientation of the laser control PCB, and finally the assembly shown at top left. Note the orientation of the 16-pin connector relative to the cutout in the manifold base. The wires extending from the laser modules pass through the cutout, across the bottom of the manifold base, and into the 16-pin connector.

At bottom left, the assembly is completed, the laser control PCB mated to the bottom of the manifold base via installation of the four pan head screws into the four press-fit threaded inserts. At bottom right, an optional battery pack is shown, consisting in one embodiment of the present invention three battery cells arrayed side by side (0.4 inches in diameter each and two inches long). The battery pack is centered widthwise behind the laser control PCB, 0.2 inches from the back of the manifold base. Battery packs such as described herein are commonly available, either off-the-shelf or custom made, which are commonly wrapped together, their power output routed via two wires to a common connector, and in this case potentially specifically made to attach to the power pins of the laser control PCB. In a portable application of the laser assembly of FIG. 28, batteries may provide a portable functionality. In one final embodiment of the present invention, the laser control PCB is connected via the 4-pin connector to an external device which provides both power and communication signals to the laser control PCB and which also provides lumbar traction or decompression simultaneously.

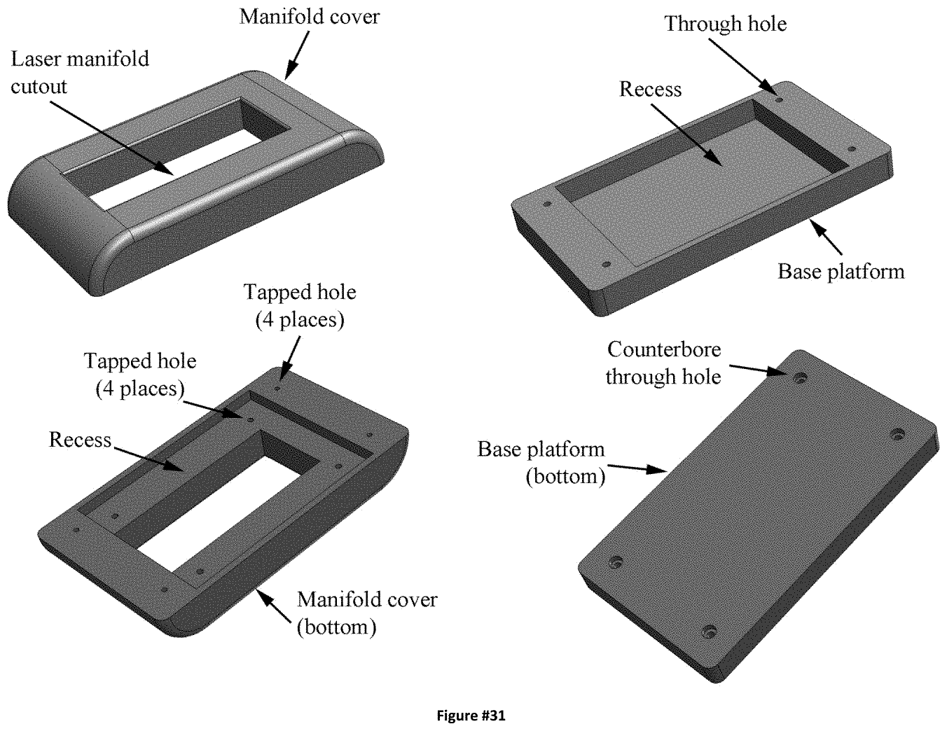

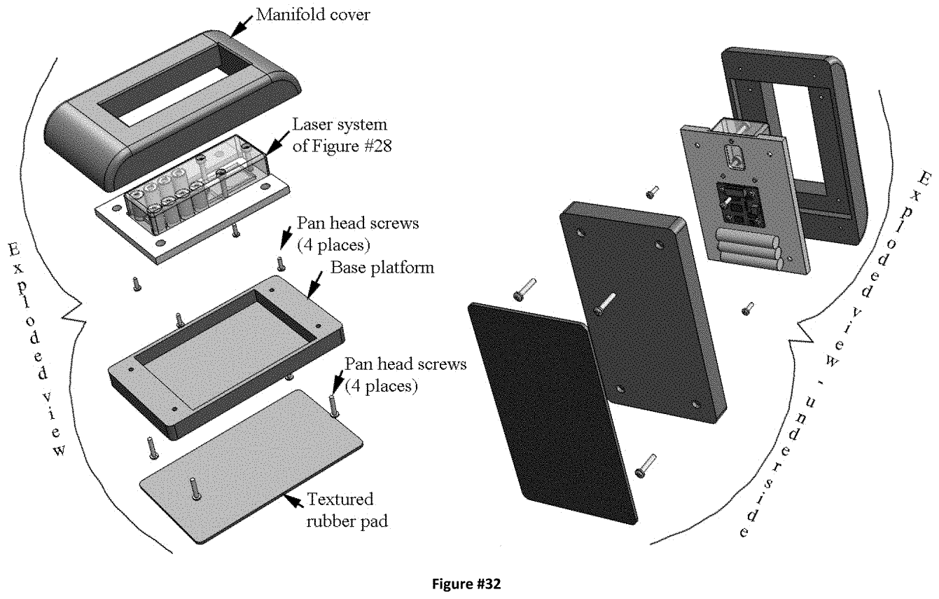

FIG. 29 describes the system of FIG. 28, with eight focused laser beams emitting from the eight laser modules installed. FIG. 30 describes the system of FIG. 28, with eight defocused laser beams emitting from the eight laser modules installed.

Although the invention has been explained in relation to its preferred embodiment(s), it is to be understood that many other possible modifications and variations can be made without departing from the spirit and scope of the invention as hereinafter claimed.

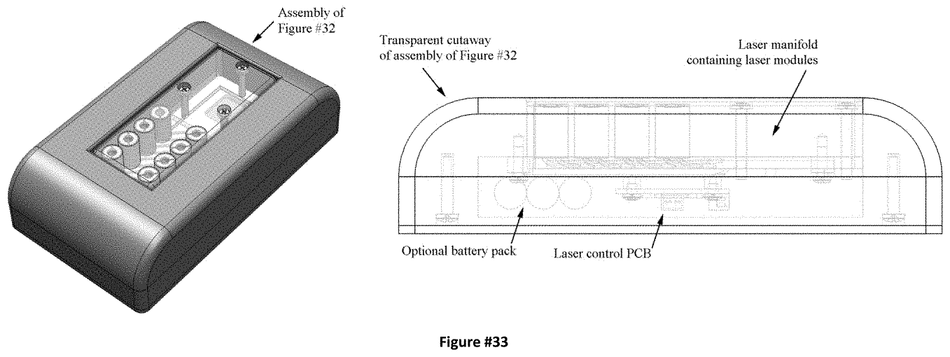

FIG. 31 describes components which can be utilized to form a portable housing for the laser system of FIG. 28. At top left, the ‘manifold cover’ is a cover upon which the laser system may be covered. In one embodiment of the present invention, the manifold cover is machined from Delrin or other non-metallic material, or may be injection molded plastic, preferably a non-metallic material. When viewed from the top, a cutout region exists allowing the top of the laser manifold to fit such that the top of the laser manifold is flush with the flat top of the manifold cover. At bottom left, the bottom of the manifold cover is shown. A secondary recession is shown, which allows the manifold base to fit flush with the bottom of the manifold cover. The bottom of the manifold cover in this embodiment of the present invention contains four tapped holes, about the outside of the bottom of the cover (in this embodiment 4-40 thread), and four tapped holes within the secondary recession (again in this embodiment 4-40 thread).

At top right, the ‘base platform’ is shown, made of similar material and manufacturing process as the manifold cover. Four through holes are shown (which are part of the four counterbore through holes shown at bottom right), as well as a recess appropriately deep to allow the laser control PCB and optional battery to reside. In this embodiment of the present invention, the manifold cover is 0.98 inches tall, 6.71 inches long and 3.8 inches wide. In this embodiment of the present invention, the base platform is 0.6 inches tall, 6.71 inches long and 3.8 inches wide, the recess being 0.5 inches deep.

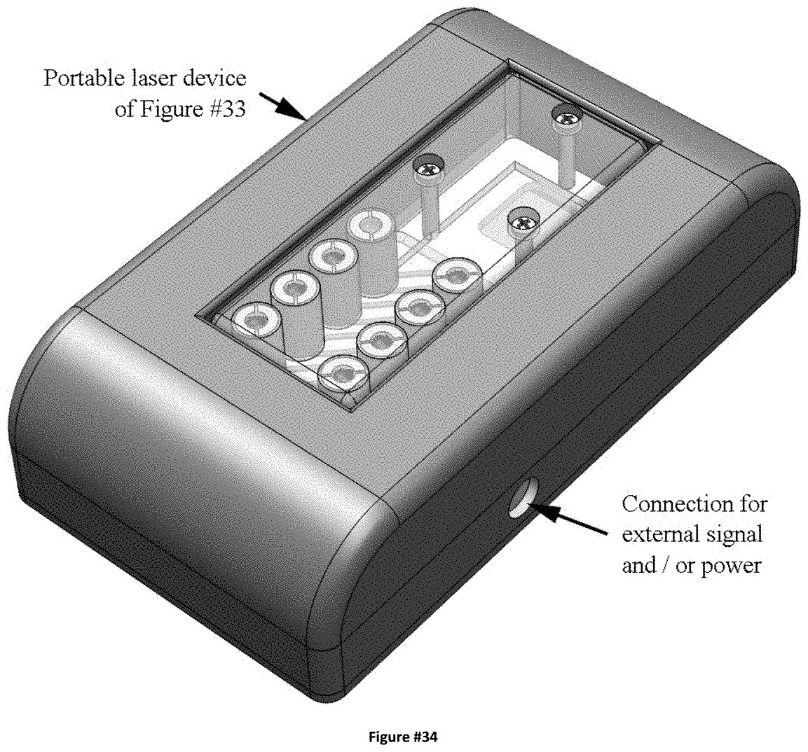

FIG. 32 illustrates exploded views of the laser system of FIG. 28 being secured into the shell formed of the manifold cover and base platform. At right, the manifold cover is shown positioned over the laser system of FIG. 28 properly oriented. Four cross-slotted pan head screws (4-40 thread, 0.375 inches long) are shown beneath the laser system of FIG. 28 which secures the laser system into the manifold cover. The manifold base then encases the manifold cover/laser system from below, secured to the manifold cover by four cross-slotted pan head screws (4-40 thread, 0.75 inches long). An optional textured rubber pad is shown, in this embodiment made of a textured silicone rubber 0.1 inches thick. This textured rubber pad may be secured via adhesive. At left, the system is shown from below. It should be noted that other embodiments of the present invention may utilize Velcro strips in place of or in addition to the textured rubber pad, and in another embodiment magnets (such as small rare-earth magnets) may be utilized in place of or in addition to the textured rubber pad for attachment to a metallic surface suitable to magnetic attraction.

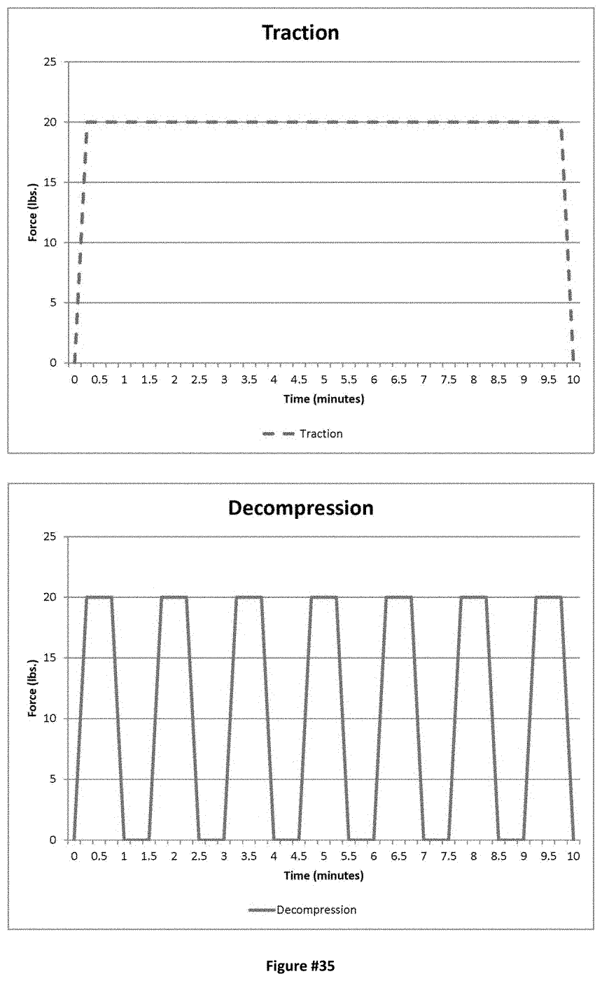

FIG. 33 shows the system of FIG. 32 fully assembled at left, the ‘portable laser device’. At right, a side view of this system is shown, in transparency such that the inner structures can be visualized within the assembly.

The portable laser device as described in FIG. 33 may be used in any number of environments. It may be used as a standalone laser therapy for topical wound healing, or may be placed for standalone lumbar or lumbar spine treatment.

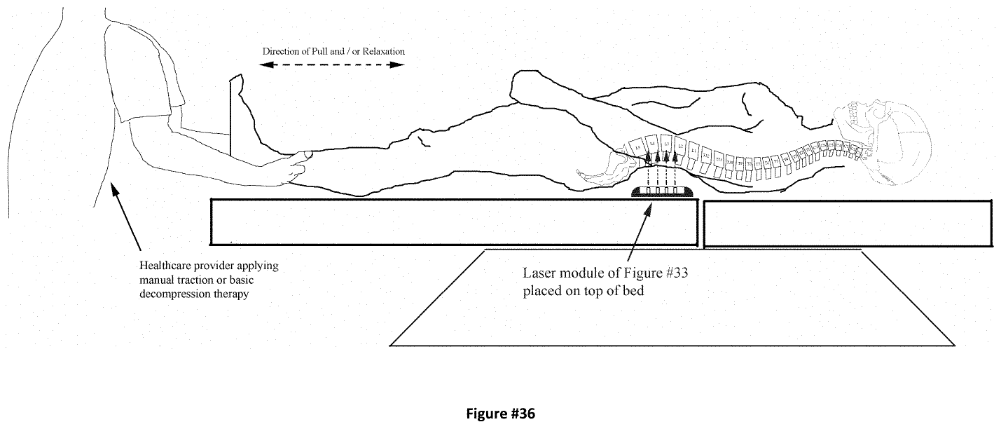

As described in FIG. 27, the laser control PCB contains a 4-pin connector designed to receive power and signal. FIG. 34 shows the portable laser device of FIG. 33, with a modification of a port or connection by which a signal may be delivered from outside of the portable laser device. In the simplest case, this may take the form of a simple on/off switch (requiring only two wire connections to two of the pins of the 4-pin connector of the laser control PCB, the other two pins of this connector being connected to the optional battery pack) such that the healthcare provider can control the laser emissions during therapy. In another form, this port may contain a connector for both external power and communications. In the case of external power and communications, the optional battery pack is not required, reducing weight and build complexity, or is contained within the portable laser device and the external power supplied may recharge the batteries. When the portable device is connected to an external system which controls an automated traction or decompression function, the external system may then have control over when the laser emissions occur, and may coordinate these during traction or decompression to achieve either lumbar laser traction or lumbar laser decompression if the two modalities are applied simultaneously.

FIG. 35, referenced previously in this provisional patent application, demonstrates an example of the profile of the forces applied during both traction and decompression therapy. At top, a graph titled ‘Traction’ shows distraction forces applied during traction (dashed line), ramping up at start of treatment and being maintained throughout the therapeutic session (y-axis is force in pounds [lbs.], x-axis is time in minutes). At bottom, a graph titled ‘Decompression’ shows distraction forces applied during either basic or true decompression. These forces ramp up at the start of treatment, are maintained for a short period of time, and then cycled down (to either a lesser distractive force or to no distractive force), the pattern repeating throughout the treatment. For either lumbar laser traction or lumbar laser decompression, the laser emissions may be on throughout the entire treatment, may be on only when the peak distractive forces in the case of either basic or true decompression are being applied, or in any combination thereof. Additionally, the lasers may operate in a continuous wave mode, either on or off, or the lasers may be pulsed at any appropriate frequency.

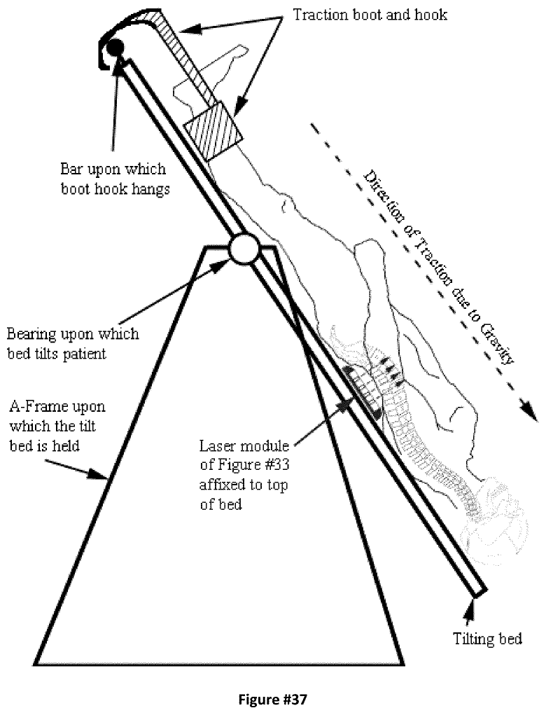

FIG. 36 represents the system of FIG. 4, with the addition of the laser module of FIG. 33. In this embodiment of the present invention, the laser module is placed underneath the patient spine, the laser beams shown as dashed arrows radiating towards the lumbar spine. The healthcare provider is applying either manual traction or manual basic decompression therapy. The laser module may be controlled (or running an independent internal program) such that it is synchronized with the traction or decompression.

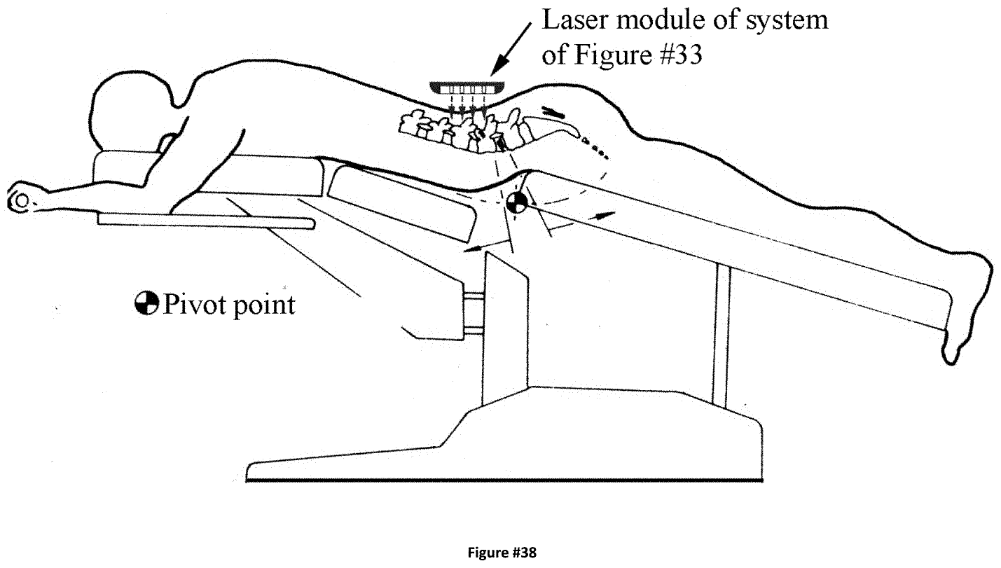

FIG. 37 represents the system of FIG. 6, with the addition of the laser module of FIG. 33. In this embodiment of the present invention, the laser module is placed underneath the patient spine, the laser beams shown as dashed arrows radiating towards the lumbar spine. The patient is shown on a rotational traction device oriented such that gravitational forces are providing traction to the spine. Given the angled position of the platform supporting the patient, the laser module may require a means of attachment to the platform. In this embodiment, the bottom of the laser module may utilize Velcro or magnets, matching a location on the platform suitable to lumbar laser irradiation. The laser module may be controlled (or running an independent internal program) such that it is synchronized with the traction or decompression.

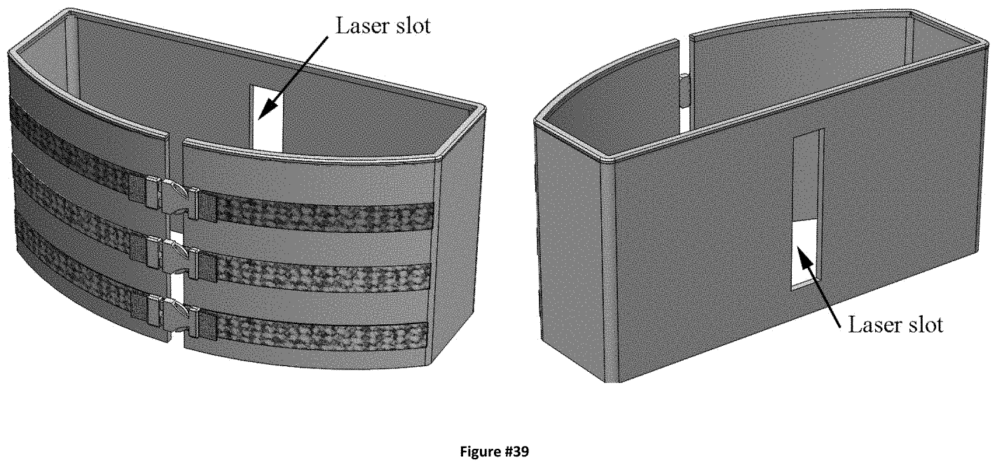

FIG. 38 represents the system of FIG. 7, with the addition of the laser module of FIG. 33. In this example, the laser module is placed on top of the lumbar spine to facilitate laser irradiation there. The laser module may be controlled (or running an independent internal program) such that it is synchronized with the traction or decompression.

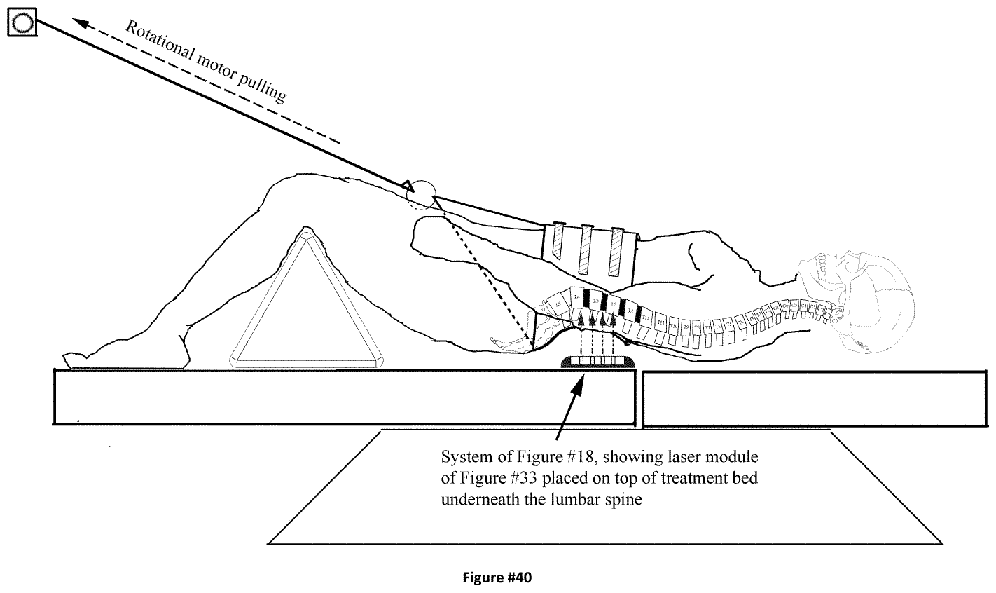

FIG. 39 represents a portion of the pelvic harness of FIG. 8, with the addition of a ‘laser slot’ in the rear of this portion. In this embodiment of the present invention, a cutout is shown which may facilitate improved penetration of laser radiation from a device such as the laser system of FIG. 33. The cutout may be a literal cutout, or may contain a window of a material suitably transparent to the wavelengths utilized by the laser system employed.

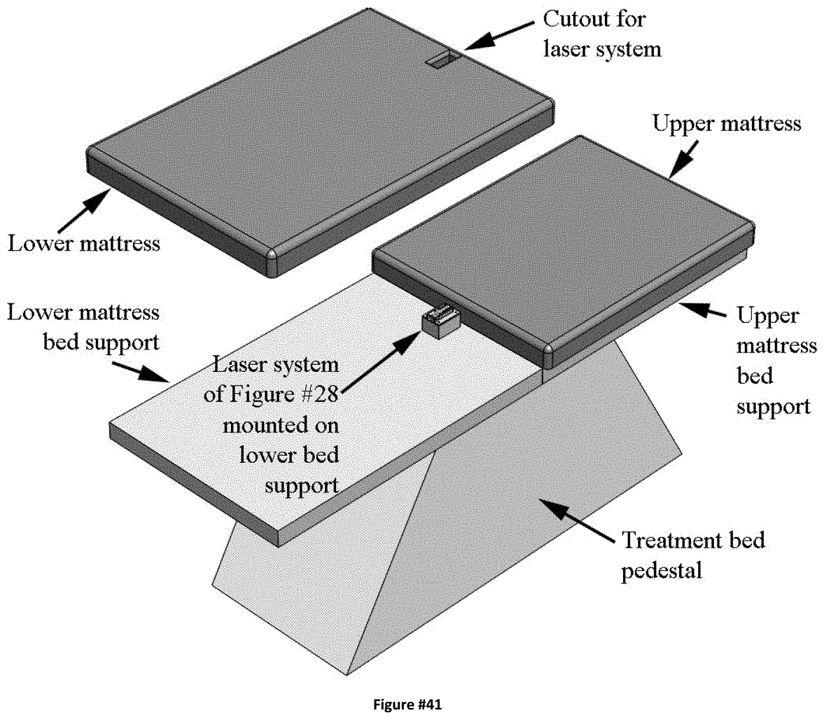

FIG. 40 represents the system of FIG. 18, with the addition of the laser system of FIG. 33. In this example, the laser system is placed on top of the treatment bed, underneath the patient's lumbar spine. The pelvic harness utilized in this embodiment may either be that of FIG. 8, or may be that described above, in FIG. 39. The laser module may be controlled (or running an independent internal program) such that it is synchronized with the traction or decompression.

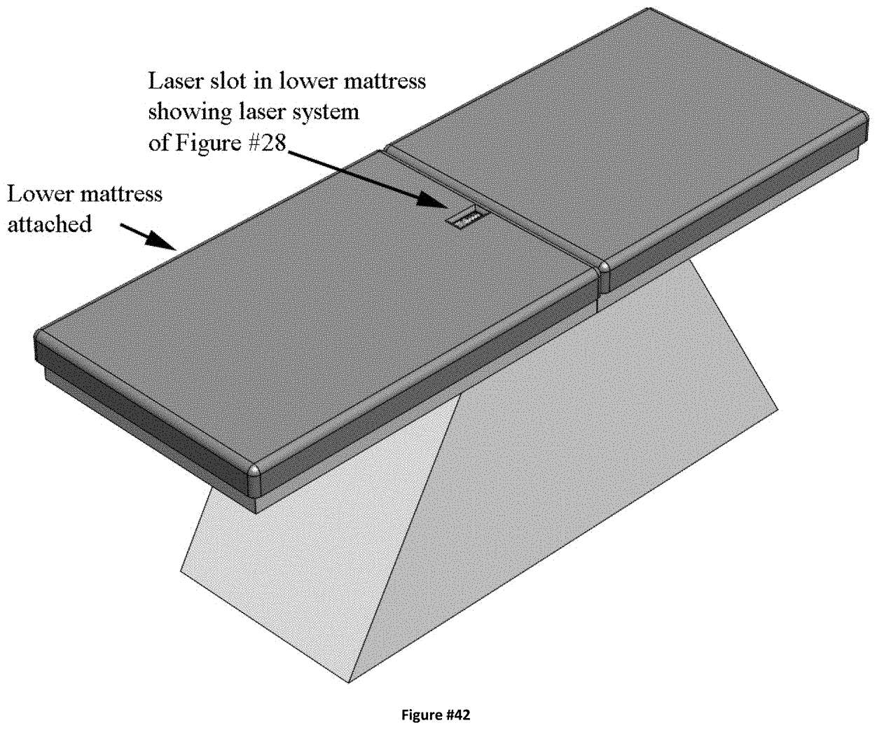

FIG. 41 depicts the treatment bed, with upper and lower mattresses, utilized in several of the previously described figures of this patent application. In this illustration, the laser system of FIG. 28 is shown mounted to the lower mattress bed support on top of a block (made of a suitably strong material, such as an aluminum or delrin for example). The height of the block positions the top of the manifold of the laser system such that, upon mounting of the lower mattress onto the lower mattress bed support, the top of the laser manifold would reside slightly below the surface of the lower mattress. The laser manifold (containing the individual laser modules in one embodiment of the present invention) is in a fixed position relative to a modification of the lower mattress in this embodiment. The modified lower mattress contains a cutout for a ‘laser slot’, which is positioned such that the laser system of FIG. 28 mounted within the treatment bed can deliver laser radiation from within the assembled treatment bed and into the patient spine (e.g. the lumbar spine).

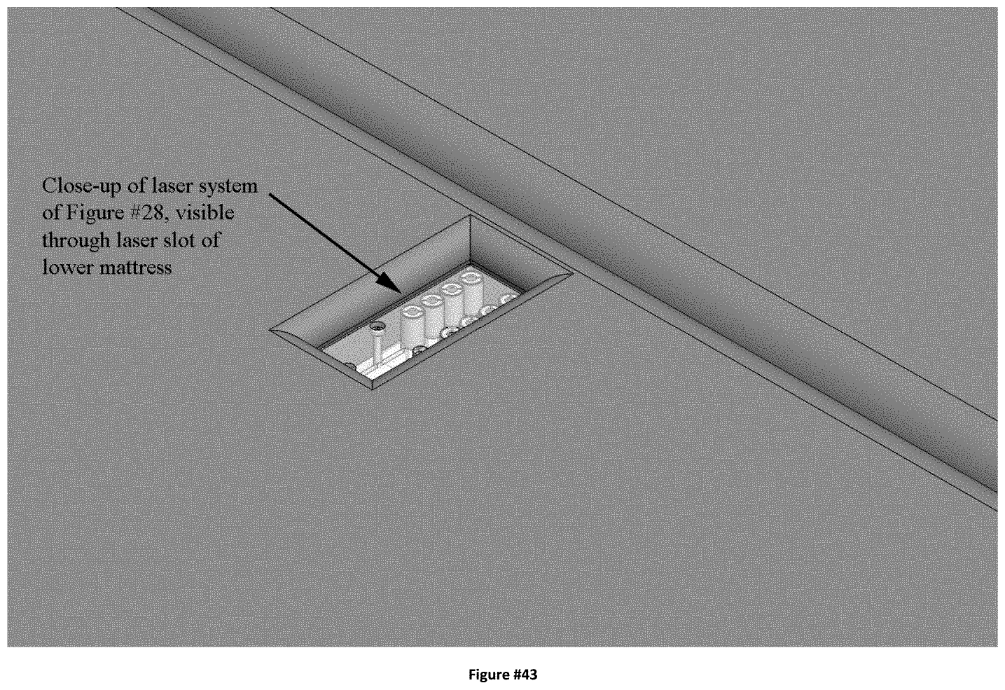

FIG. 42 represents the system of FIG. 41, with the lower mattress secured to the lower mattress bed support. The laser manifold of the mounted laser system of FIG. 28 is clearly visible within the laser slot described in FIG. 41.

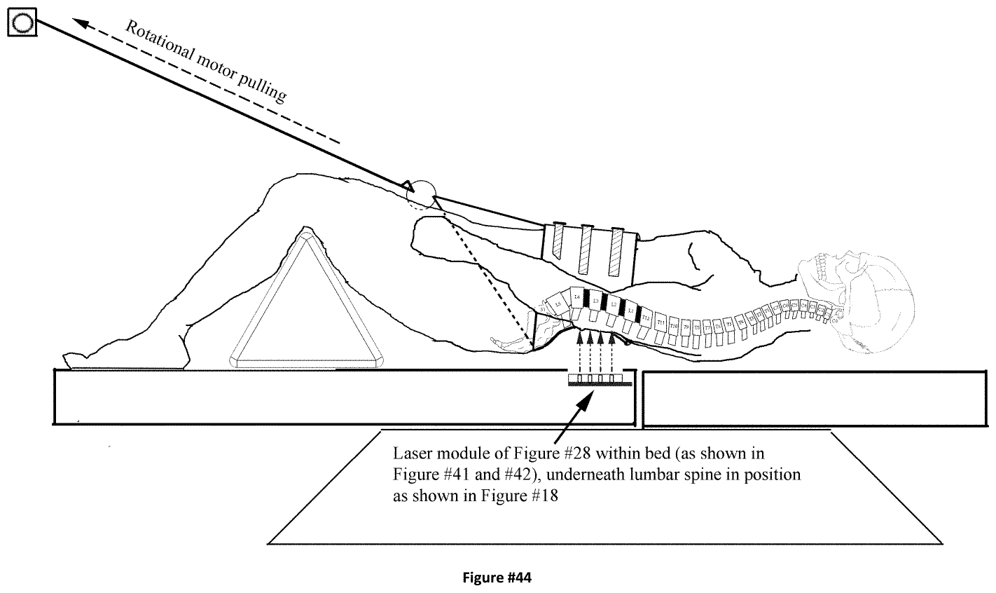

FIG. 43 is a close-up of FIG. 42, centered on the laser slot and laser system of FIG. 28. This illustration further clarifies the access of the laser system to irradiate the patient from within the treatment bed.

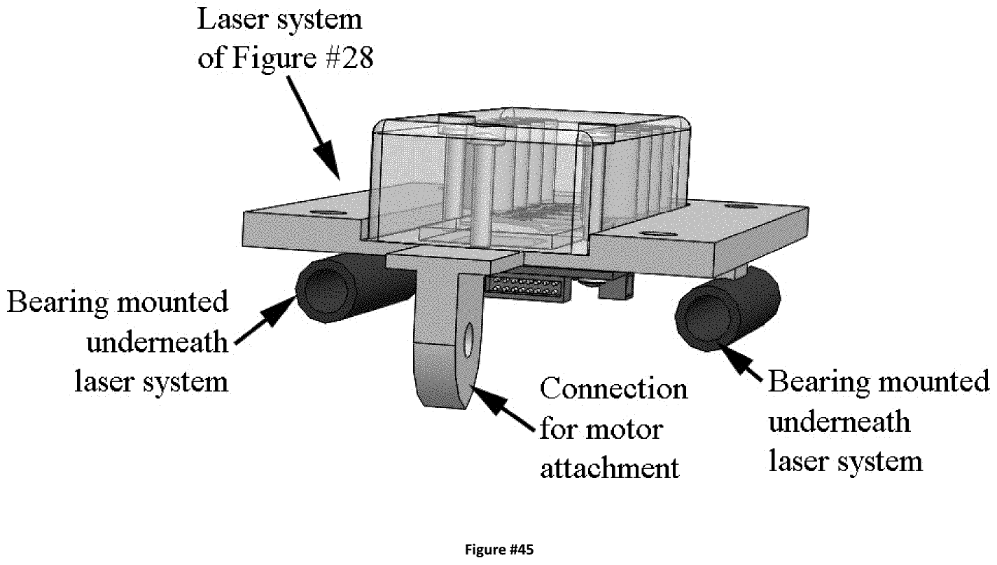

FIG. 44 represents a patient in traction or decompression as was described in FIG. 18. The treatment bed is the modified treatment bed of FIG. 42, with the laser system of FIG. 28 mounted within the lower mattress and irradiating the patient's lumbar spine. In this embodiment, the patient is preferably harnessed utilizing the modified pelvic harness with the laser cutout/window of FIG. 39. The laser module may be controlled (or running an independent internal program) such that it is synchronized with the traction or decompression.

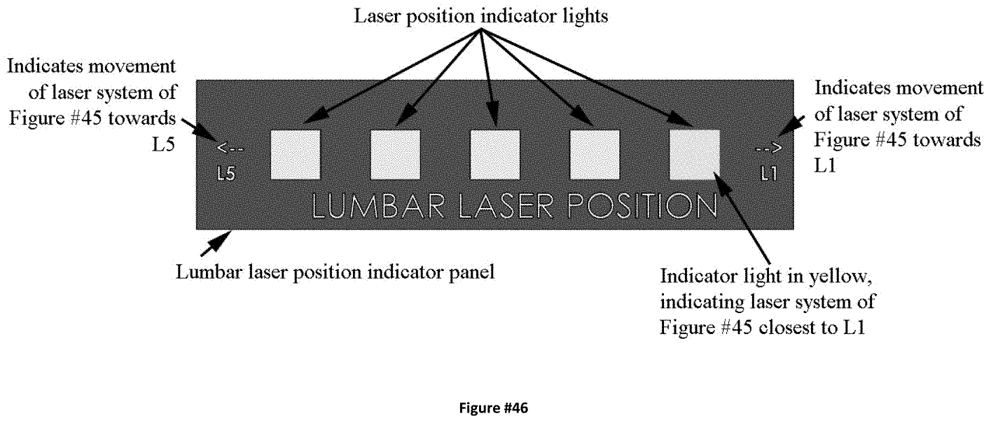

FIG. 45 represents a modified version of the laser system module of FIG. 28. In this embodiment of the present invention, the manifold base contains small mounts upon which bearings (e.g. Teflon or ball bearings to be mounted on rods) are secured (shown in red) in two places. The manifold base is further modified to include an attachment point for some form of automated means, such as a rotational motor attached by strap or cord, a stepper motor, or a linear actuator for example.

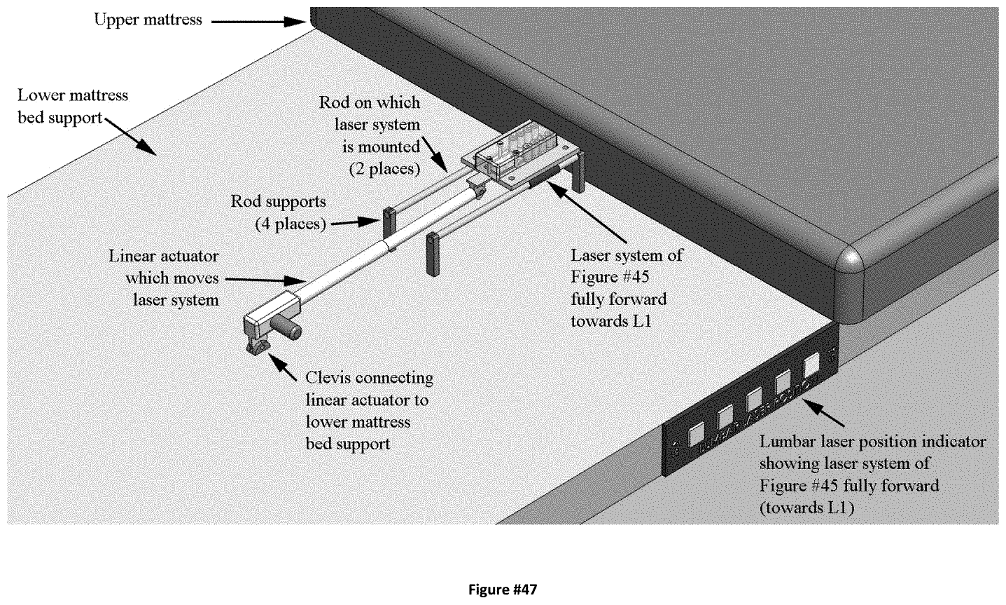

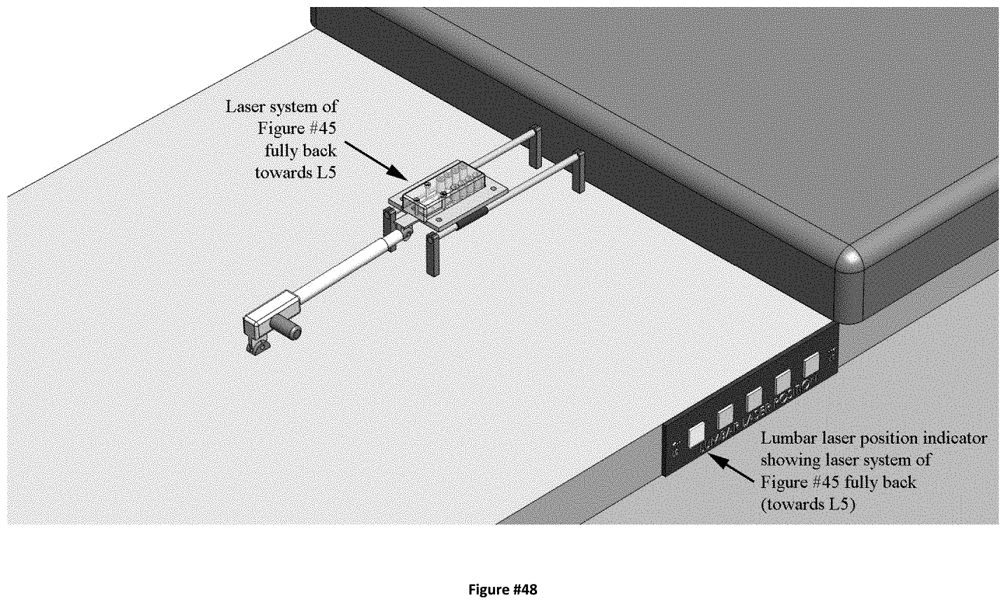

FIG. 46 represents one embodiment of the present invention which may be utilized to indicate the position of the laser system of FIG. 45. As will be shown and described in FIGS. 47 through 51, the laser system of FIG. 45 is mounted within the treatment bed such that it can slide forwards towards the L1 vertebra of the patient, or backwards towards the L5 vertebra of the patient. This action may be accomplished via a program contained within the laser system itself, or may be controlled externally, allowing a healthcare provider to radiate closer to an intended lumbar region of the spine. The ‘Lumbar Laser Position’ indicator, in this embodiment a panel, contains five squares which may be backlit. It may be in direct communication with the laser system of FIG. 45, or in communication with an external means, such that one of the five squares is illuminated indicating the position of the laser system within the treatment bed. In this illustration, the square shaded in yellow indicates the laser system is fully forward, closest to the L1 vertebra location. In some instances, if the laser system is capable of delivering very intense laser radiation, safety regulations may require safety features in a laser emitting device which indicate the target of the laser radiation. If the wavelengths of the laser radiation are not visible, and if a visible targeting device would be rendered useless via the application in which the laser system is employed (as in one embodiment of the present invention, fully underneath a patient and not visible to a healthcare provider), the lumbar laser position panel may satisfy the needs of such safety regulations.

FIG. 47 represents a modified treatment bed in one embodiment of the present invention. In this embodiment, the laser system of FIG. 45 is mounted atop two rods (those rods passing through the bearings of the laser system) which may be made of a suitable material such as stainless steel, aluminum, Teflon, Delrin, plastic, etc. The two rods are secured in four rod supports (which may be an injection molded material or machined material suitable to the application). An automated means of pulling or pushing the laser system of FIG. 45 is shown, in this example a linear actuator motor, connected at one end to the laser system and at the other end to a clevis attached to the lower mattress bed support. There are many means of determining motor position, in this example one means employed may be an encoder installed within the linear actuator and in communications with either the laser control PCB or an external control means. The motor may be in communication with a controlling means which ensures the laser system travels only within a certain range upon the rods. In one embodiment, the encoder information may also be used to communicate with the lumbar laser position panel to indicate where the laser system is positioned. In this embodiment the laser system is shown fully forward (towards the L1 region), and the laser position indicator panel displays a lit position indicator for the fully forward position.

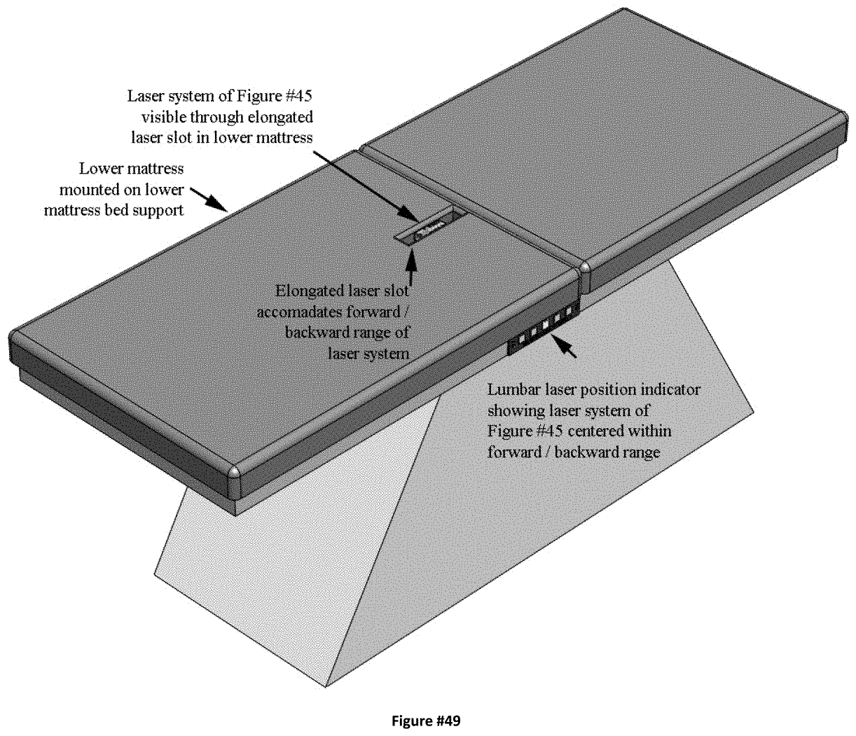

FIG. 48 is the system of FIG. 47, where the laser system of FIG. 45 has been moved backwards fully towards the L5 region (as also indicated by the lit indicator on the lumbar laser position panel).

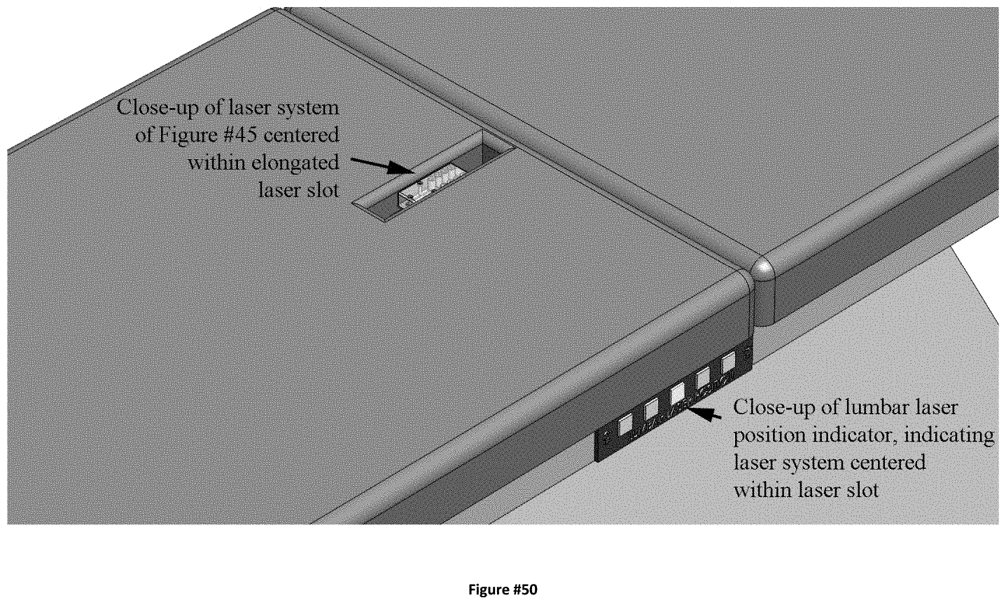

FIG. 49 is a full view of the system of FIGS. 47 and 48, with the addition of a modified lower mattress mounted to the lower mattress bed support. In this embodiment, the modified lower mattress contains an elongated laser slot, suitable to providing access to the laser system of FIG. 45 within the full range of movement towards either the L1 or L5 regions of the spine. In this illustration, the laser system is positioned mid-range, and the lumbar laser position indicator indicates this mid-position via the yellow lit indicator.

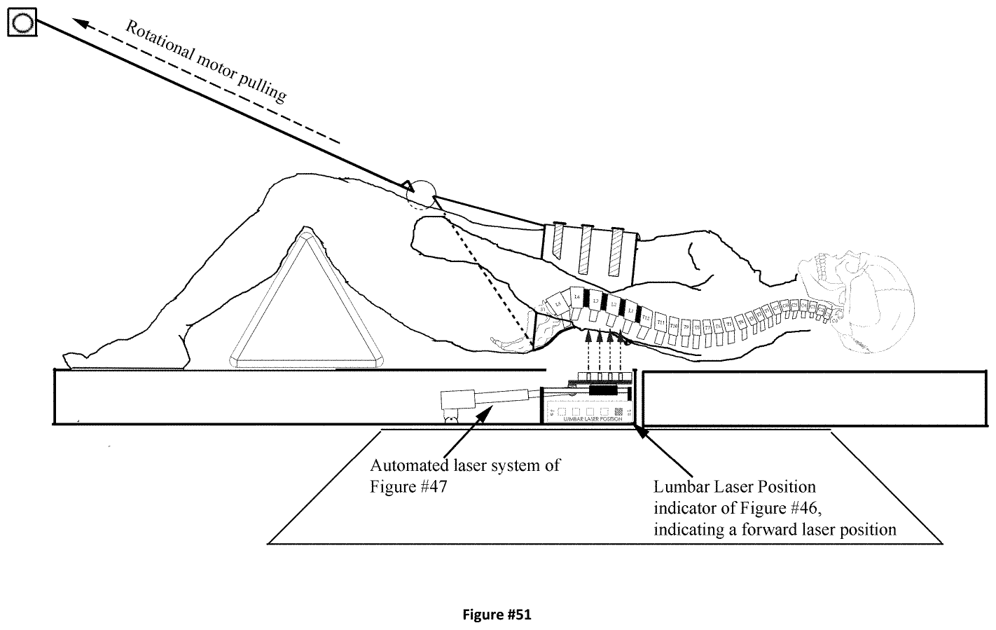

FIG. 50 provides a close-up view of the system of FIG. 49. The close-up focuses on the laser system of FIG. 45, centered mid-range within the elongated laser slot of the modified lower mattress. A close-up view of the lumbar laser position panel shows the middle indicator lit in yellow, corresponding to the laser system's position.

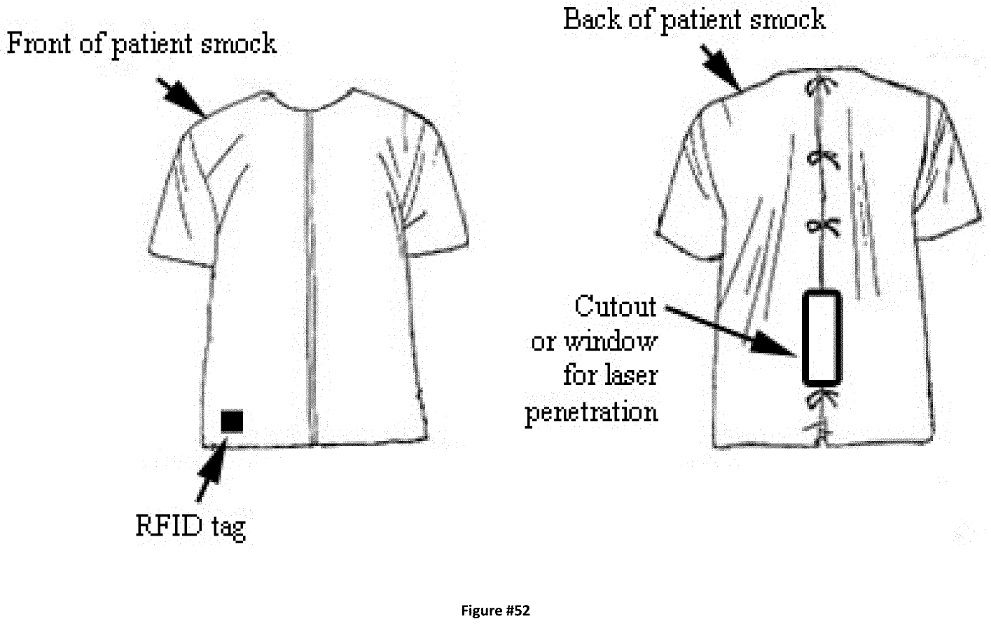

FIG. 51 represents one preferred embodiment of the present invention. The patient is supine on the treatment bed, the treatment bed being that of FIG. 49 containing both the automated sliding laser system and the lumbar laser position panel (said panel being mounted on the side of the lower mattress bed support such that the five light indicators correspond to the full range of the laser system's slide range). In this embodiment, the patient is preferably harnessed utilizing the modified pelvic harness with the laser cutout/window of FIG. 39. The rotational motor is connected to the ring of the pelvic harness via a strap or cord, such that the intervertebral discs targeted for true decompression therapy are aligned. Through the application of cyclical tension via the rotational motor, the intervertebral discs which have been aligned are elongated under tension.

A side view of a cut away of the laser system of FIG. 37, which facilitates the movement and preferred positioning of the laser emissions relative to the region of interest of the lumbar spine is shown within the treatment bed. The lumbar laser position panel is shown mounted to the lower mattress bed support, and in this figure the indicator light on the panel corresponding to a fully forward position (towards L1) is shown (with hash marks). In this figure, the laser manifold containing the lasers is moved fully forward towards the L1 position. In this embodiment, the elongated intervertebral discs of interest are shaded in black, and are receiving laser therapy during the true decompression session. The laser module may be controlled (or running an independent internal program) such that it is synchronized with the traction or decompression.

One additional consideration of the present invention contemplates the utilization of a modified patient gown, as may be seen commonly in hospitals and clinical settings. Refer to FIG. 52. The front and back of a typical patient gown is shown (front side at left, back side at right). At right, the back of the patient gown or smock is shown containing a cutout located approximately along the lumbar spine. The cutout also is appropriately sized and located to accommodate a laser emission system as has been described previously, as in FIG. 51 for example. The cutout may simply be a literal cutout, or may contain a window, the material of which suitably transparent to the desired laser wavelengths. In one consideration, the patient smock may further be impregnated with an antimicrobial agent, such as a silver compound, for additional hygiene. This is a common practice in many medical bandage applications.

The modified patient gown may contain a device which may authorize traction or decompression laser therapy sessions, track treatment sessions, or any number of other metrics. In one embodiment of the present invention, this device may take the form of a radio frequency identification tag (RFID tag). RFID tags are read wirelessly and are found commonly in medical practice. RFID tags are commonly found sewn into clothing, and in this case the modified patient gown or smock. In one embodiment of the present invention, the RFID tag may be read by a device within the treatment bed, by a device contained within the housing/control system for the lumbar laser decompression, or via any number of other means suitable to the application of the present invention. The RFID tag may serve as an additional safety feature, ensuring only patients who have been authorized for laser decompression (or traction) therapy receive said therapy.

The foregoing summary, as well as the following detailed description of certain embodiments of the present invention, will be better understood when read in conjunction with the appended drawings. For the purpose of illustrating the invention, there is shown in the drawings certain embodiments. It should be understood, however, that the present invention is not limited to the arrangements and instrumentalities shown in the attached drawings. For example, while lasers have been described extensively in the application, many forms of light emitting devices are readily available. Light emitting diodes (LEDs) may be utilized in place of lasers, and while the light emitted is not necessarily coherent or collimated, collimating lenses are readily utilized to collimate LED light for the purpose of light therapy. Further, for the purposes of light therapy, little difference has been demonstrated clinically comparing the use of lasers or LEDs for light therapy.

1. A laser therapy device, comprising:
a laser manifold having a plurality of recessions therein; and
a plurality of laser modules, each disposed within one of the plurality of recessions in the laser manifold.
